(19) United States
(12) Patent Application Publication
(10) Pub. No.: US 2024/0269673 A1
(43) Pub. Date: Aug. 15, 2024

(54) CENTRIFUGAL MICRO-FLUIDIC DISK FOR DETECTING THE PRESENCE AND CONCENTRATION OF AN ANALYTE OF INTEREST

(71) Applicant: LIFELOC TECHNOLOGIES, INC., Wheat Ridge, CO (US)

(72) Inventors: Charles A. GONZALES, Wheat Ridge, CO (US); Curtis H. STUMPF, Wheat Ridge, CO (US); Wayne Robert WILLKOMM, Wheat Ridge, CO (US)

(21) Appl. No.: 18/440,798

(22) Filed: Feb. 13, 2024

Related U.S. Application Data
(60) Provisional application No. 63/484,706, filed on Feb. 13, 2023.

(51) Int. Cl. B01L 3/00; G01N 35/00; G01N 35/04

(52) U.S. Cl. CPC B01L 3/502753; B01L 3/5021; B01L 3/502715; B01L 3/502761; G01N 35/00; B01L 2200/0652; G01N 2035/00574; G01N 2035/0449

(57) ABSTRACT

The presently disclosed technology may utilize micro-fluidic disk technology, including integrated reagent and/or control one-time use fluid packs that improve functionality and ease of use of the presently disclosed disk. In various implementations, fluids within the disk may move radially inward as well as radially outward as required to functionally achieve the methods described herein. The presently disclosed technology may also be used to distinguish an analyte of interest from the background noise of one or more metabolites thereof in a fluid sample. This may be achieved by magnetically pre-separating some or all metabolites from the analyte of interest within the fluid sample. Using the disclosed techniques, detection of the analyte of interest is not obscured by other compounds leading to false positive test results.

### CROSS-REFERENCE TO RELATED APPLICATIONS

The present application claims benefit of priority to U.S. Provisional Patent Application No. 63/484,706 entitled “Magnetic Pre-Separation in Sample Assay for Evaluating User Intoxication” and filed on Feb. 13, 2023, which is specifically incorporated by reference herein for all that it discloses or teaches.

### BACKGROUND

State-level legalization in the United States of America of marijuana (e.g., recreational and/or medicinal use), has yielded varying laws that regulate the permissible levels of (−)-trans-Δ⁹-tetrahydrocannabinol ((6aR,10aR)-delta-9-tetrahydrocannabinol) (also referred to herein as delta-9-THC, delta-9, and Δ9) in a subject's blood. There are, however, few screening methods amenable to road-side law enforcement that are specific to delta-9-THC.

Delta-9-THC is the active ingredient in marijuana capable of causing intoxication and is therefore the target of interest for intoxication screening. Marijuana users have detectable levels of tetrahydrocannabinol-based metabolites in their system, including delta-9-THC. Delta-9-THC is rapidly converted in a user's body to other THC forms, such as hydroxy-THC and carboxy-THC. As the many forms of THC are lipophilic, these compounds are stored in a user's fat cells and will gradually eliminate from the user's body over the course of several weeks. The short timeframe conversion of delta-9-THC compounds to various metabolites, to the long timeframe elimination of the metabolites from a habitual user's body, often results in a small volume of delta-9-THC being present in the user's body as compared to a higher relative volume of metabolites also being present.

Current diagnostic screening tests (typically performed in urine for workplace drug testing) utilize reagents that detect all forms of THC. In jurisdictions where marijuana is illegal this is an extremely convenient consequence, as significant levels of THC and its many metabolites and compounds will be available for screening. However, in jurisdictions where marijuana is legal, any non-delta-9-THC-based metabolites can interfere with accurate detection of levels of any delta-9-THC-based metabolites and compounds in an individual's bloodstream. Further, in a habitual user's blood, the quantity of metabolites can be thousands of times greater than an amount of delta-9-THC.

Still further, the problem of detecting delta-9-THC is exacerbated by the relatively low levels of delta-9-THC in the bloodstream that constitute intoxication under various state laws. For example, as of the date of this writing, six US states (i.e., Colorado, Illinois, Montana, Nevada, Ohio, and Washington) have set specific limits for an individual to be considered intoxicated based on the level of delta-9-THC in the individual's bloodstream. These limits range between 2 nanograms per milliliter (ng/mL) and 5 ng/ml of blood depending on the state.

### SUMMARY

Implementations described and claimed herein address the foregoing problems by providing a centrifugal micro-fluidic disk comprising an array of chemical detection sectors, each extending radially outward from a sample input port. Each of the chemical detection sectors includes a metering chamber to receive a test subject sample from the sample input port and meter a quantity of the test subject sample out of the metering chamber, an antibody mixing chamber to mix the metered test subject sample with antibodies having regions specific to an analyte of interest and a fluorophore attached thereto, a high-density bead chamber containing high-density beads each with a first protein that is specific to the analyte of interest, the high-density beads further with an analyte of interest simulant molecule attached thereto, the high-density bead chamber to mix the metered test subject sample and antibodies with the high-density beads, and a detection chamber to receive the metered test subject sample, antibodies, and high-density beads, wherein antibody combinations with fluorophores attached are separated from the high-density beads using a centrifugal force, and wherein an absence of fluorescence from the high-density beads indicates a presence of the analyte of interest within the fluid sample.

Other implementations are also described and recited herein.

### BRIEF DESCRIPTION OF THE DRAWINGS

FIG. 1 illustrates an example centrifugal micro-fluidic disk with an integrated reagent fluid pack and an integrated control fluid pack that may be used to analyze a test subject's fluid sample for detecting the presence of one or more analytes of interest referenced against one or more control sample measurements.

FIG. 2 is a functional schematic of an example centrifugal micro-fluidic disk with integrated reagent that may be used to analyze a test subject's fluid sample for detecting the presence of one or more analytes of interest referenced against integrated high and low control assays measurements.

FIG. 3 is a functional schematic of a singular drug detection sector within a centrifugal micro-fluidic disk.

FIG. 4 is a functional schematic of an integrated reagent sector within a centrifugal micro-fluidic disk.

FIG. 5 is a schematic of a centrifugal micro-fluidic disk drug detection system used to determine if a fluid sample includes a drug-related compound.

FIG. 6 illustrates example additives to aid detection of delta-9-THC in a fluid sample.

FIG. 7 illustrates magnetic pre-separation sequence separating metabolites from a fluid sample including various metabolites but no delta-9-THC.

FIG. 8 illustrates a delta-9-THC detection sequence using fluorescence within a testing area of a detection chamber within a micro-fluidic disk with the magnetically pre-separated detection sample of FIG. 7.

FIG. 9 illustrates magnetic pre-separation sequence separating metabolites from a fluid sample including various metabolites and delta-9-THC.

FIG. 10 illustrates a delta-9-THC detection sequence using an absence of fluorescence within a testing area of a detection chamber within a micro-fluidic disk with the magnetically pre-separated detection sample of FIG. 9.

FIG. 11 is a flowchart of example operations for executing a magnetic pre-separation sequence on a fluid sample.

FIG. 12 is a flowchart of example operations for executing a delta-9-THC detection sequence on a fluid sample.

FIG. 13 presents an example graph of fluorescence versus concentration for various analytes of interest obtained as a result of the detection sequences described herein.

### DETAILED DESCRIPTION

Various technologies pertaining to detecting a drug-related compound utilizing portable and relatively quick assaying system are described below. While presented with specificity regarding the delta-9-THC psychoactive component of marijuana as the analyte of interest, various implementations presented herein can be directed at detecting a variety of drugs, viruses, bacteria, chemical compounds, etc. as the analyte of interest. These compounds may include, by way of example and not limitation those relating to (derived from, comprising, associated with) marijuana (e.g., tetrahydrocannabinol (THC), (−)-trans-Δ⁹-tetrahydrocannabinol ((6aR,10aR)-delta-9-tetrahydrocannabinol, delta-9-THC, Δ9-THC, etc.), methamphetamine, amphetamine, cocaine, opiates (e.g., fentanyl, heroin, opium), MDMA (3,4-methylenedioxy-methamphetamine), ketamine, PCP (1-(1-phenyl cyclohexyl) piperidine), PCP analogs, lysergic acid diethylamide (LSD), psilocybin, etc. Other example analytes of interest detected using the implementations presented herein may include performance enhancing drugs (e.g., oxandrolone, stanozolol, and erythropoietin), food pathogens (e.g., E. coli O157:H7, Listeria spp., and Salmonella spp.), water testing (coliforms, Legionella, and Enterococci), and human or veterinary health diagnostics (e.g., Streptococcus pneumoniae, Respiratory Syncytial Virus, COVID-19).

In a jurisdiction where marijuana usage is legalized, rather than attempting to detect whether a person has used marijuana at some point previously, the focus is on determining whether a person is currently under the influence of marijuana as indicated by whether the person has a higher than legal amount of delta-9-THC currently in their body. As the delta-9-THC compound metabolizes to form metabolites over time, and the metabolites are generally not psychoactive compounds, detection of whether a person is currently under the influence of marijuana requires that detection of delta-9-THC be distinguished from various metabolites of the delta-9-THC.

The presently disclosed technology may utilize centrifugal micro-fluidic disk (also referred to as centrifugal micro-fluidic biochip, lab-on-a-chip, lab-on-a-disk, or biodisk) technology to implement the systems and method described herein. Specifically, the presently disclosed technology may utilize integrated reagent and/or control one-time use fluid packs (e.g., blister packs) that improve functionality and ease of use of the presently disclosed micro-fluidic disk over prior art designs. In various implementations, fluids within the disk may move radially inward as well as radially outward as required to functionally achieve the methods described herein.

The presently disclosed technology may be used to distinguish the delta-9-THC compound from the background noise of one or more metabolites in a fluid sample (e.g., a sample of the subject's saliva, blood, plasma, sweat, vitreous humor, etc.). This may be achieved by magnetically and/or centrifugally pre-separating some or all of the delta-9-THC metabolites from the delta-9-THC within a sample. Using the disclosed techniques, detection of the delta-9-THC is not obscured by the other compounds (e.g., delta-9-THC metabolites) leading to false positive test results for a subject that previously consumed marijuana but is not currently under its psychoactive effects, for example.

A timely and accurate determination of current marijuana (or other drug) intoxication may be important prior to approving a subject to operate a machine, vehicle, etc. Such an individual may have recently smoked or otherwise consumed marijuana, and accordingly may be attempting to drive a vehicle while under the influence of delta-9-THC. This places the subject at a higher risk of causing harm to themselves or others or property damage using the machine, vehicle, etc. due to the subject's current mental impairment. The presently disclosed technology can be utilized in a law enforcement roadside stop or in a workplace as a tool to help assess the current state of an individual, or at least provide a detected concentration of delta-9-THC in the individual fluid sample referenced against a legal limit. The presently disclosed technology may be portable, provide a timely assessment (e.g., timely provide a level of delta-9-THC in a driver's fluid sample), and easy to implement by the subject, a police officer, a testing administrator, etc.

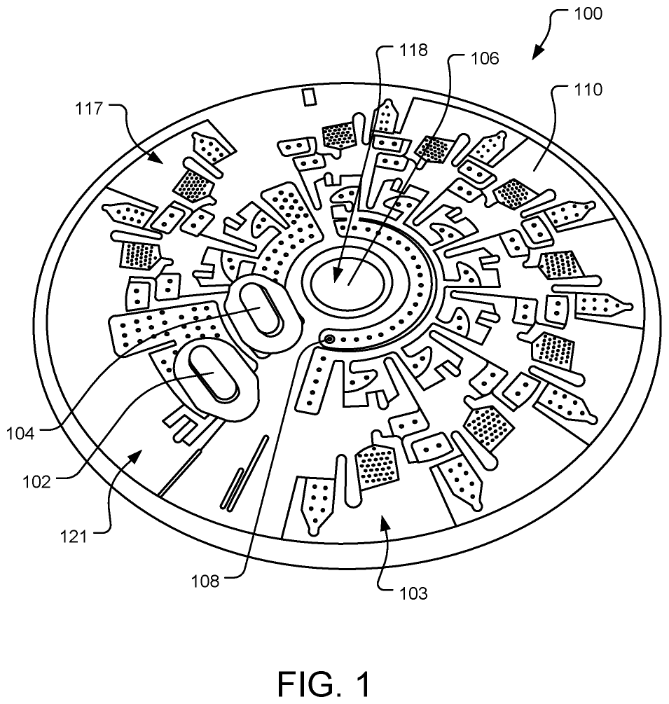

FIG. 1 illustrates an example centrifugal micro-fluidic disk 100 with an integrated reagent fluid pack 102 and an integrated control fluid pack 104 that may be used to analyze a test subject's fluid sample for detecting the presence of one or more analytes of interest referenced against one or more control sample measurements. Each of the integrated reagent fluid pack 102 and the integrated control fluid pack 104 may be single-use in that once punctured or otherwise opened thereby releasing fluid therein, the integrated reagent fluid pack 102 and the integrated control fluid pack 104 cannot be used again.

The disk 100 is a type of lab-on-a-chip technology that can be used to integrate a series of processes such as separating, mixing, reacting, and detecting into a singular platform that takes the general form factor of a spinning disk. The disk 100 utilizes microfluidics to take advantage of non-inertial pumping using non-inertial valves and switches experiencing and utilizing a balance of centrifugal, Coriolis, Euler, and other forces to distribute fluids about the disk 100 in a highly parallel order.

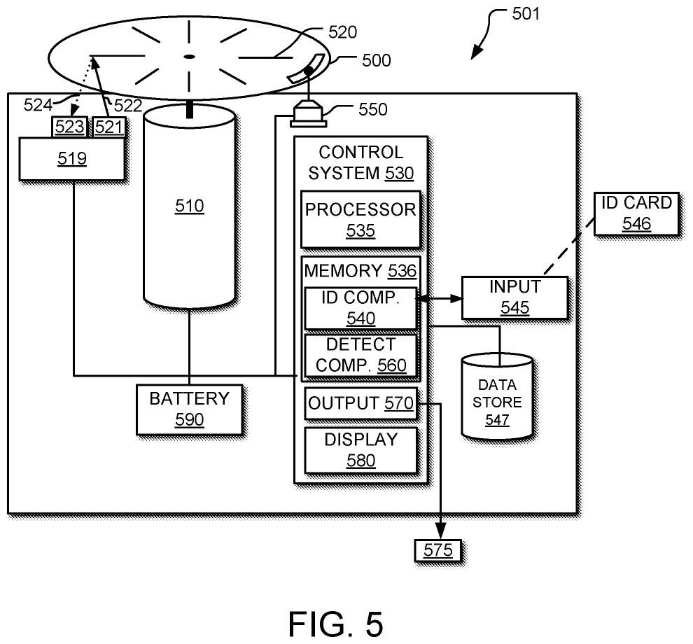

The disk 100 generally comprises a multi-layered sandwiched substrate 110 with a center 106, at which there is a locating hole 118 for positioning the disk 100 on a motor (not shown, see e.g., motor 510 of FIG. 5) to be spun. The sandwiched substrate 110 encapsulates a variety of features within the disk 100, as discussed in detail below. The substrate 110 may be formed from any suitable material, such as quartz, glass, polymethyl methacrylate (PMMA), cyclo olefin polymer (COP), polycarbonate (PC), fused silica, Polydimethylsiloxane (PDMS), or another transparent substrate that enables optical observation of samples within the disk 100. In other implementations, the disk 100 may be formed from polymers, metals, semiconductors, etc. Further, multiple materials can be utilized in combination to form the disk 100. Further, any suitable surface treatments, coatings, etc., may be utilized, to enhance compatibility with fluid(s) introduced into the substrate 110. For example, surface treatments, coatings, etc., may be utilized to control interaction of fluids with the substrate 110. While the substrate 110 is illustrated as circular in form, the substrate 110 can have any shape, size, thickness, etc., to enable any implementation presented herein (e.g., the substrate 110 can have a square profile).

The substrate 110 may define a variety of microfluidic features. Generally, microfluidic, as used herein, refers to a system, device, or feature having a dimension of about 1 mm or less (or about 500 μm or less, or about 100 μm or less) and suitable for at least partially containing a fluid. Other dimensions may be used. The substrate 110 may define one or more microfluidic features, including any number of chambers, channels, inlet/outlet ports, valves, mixers, meters, switches, etc. Further, various microscale fabrication techniques, such as embossing, etching, injection molding, surface treatments, photolithography, bonding, and other techniques, may be utilized to fabricate the disk 100.

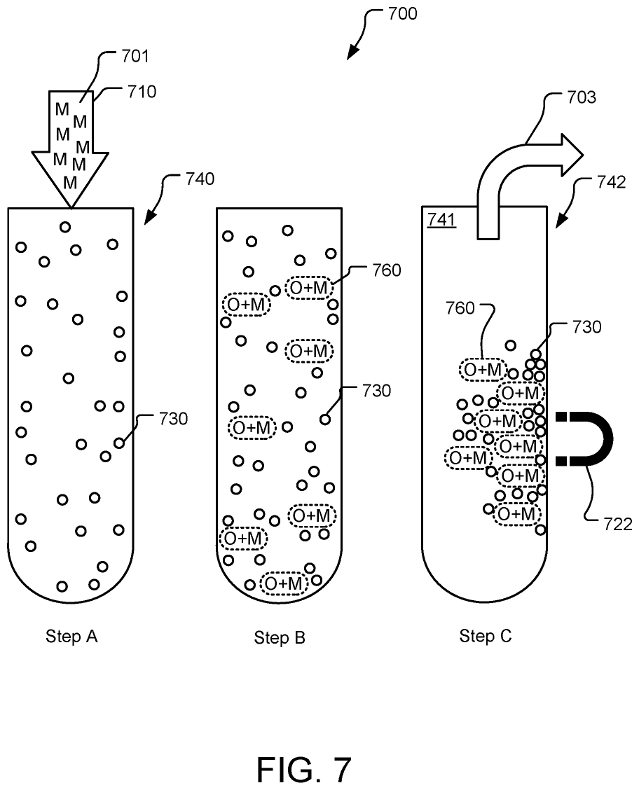

The disk 100 includes an array of sectors generally arranged radially about the disk 100. Each of the sectors are assigned a specific function within the disk 100. For example, the disk 100 includes drug detection sectors (e.g., drug detection sector 103) that are used to analyze a subject fluid sample (not shown, see e.g., subject fluid sample 710 of FIG. 7) introduced at subject fluid input 108 for the presence of various drugs or other chemicals. The disk 100 also includes control sectors (e.g., control sector 117) that are used to perform a similar analysis on a control sample released by the integrated control fluid pack 104 for comparison against the drug detection sectors. The disk 100 also includes a liquid reagent sector 121 that is used to fill or rehydrate one or more dehydrated chambers found in any or all of the drug detection sectors and the control sectors using liquid reagent released by the integrated liquid reagent fluid pack 102 and/or the integrated control fluid pack 104.

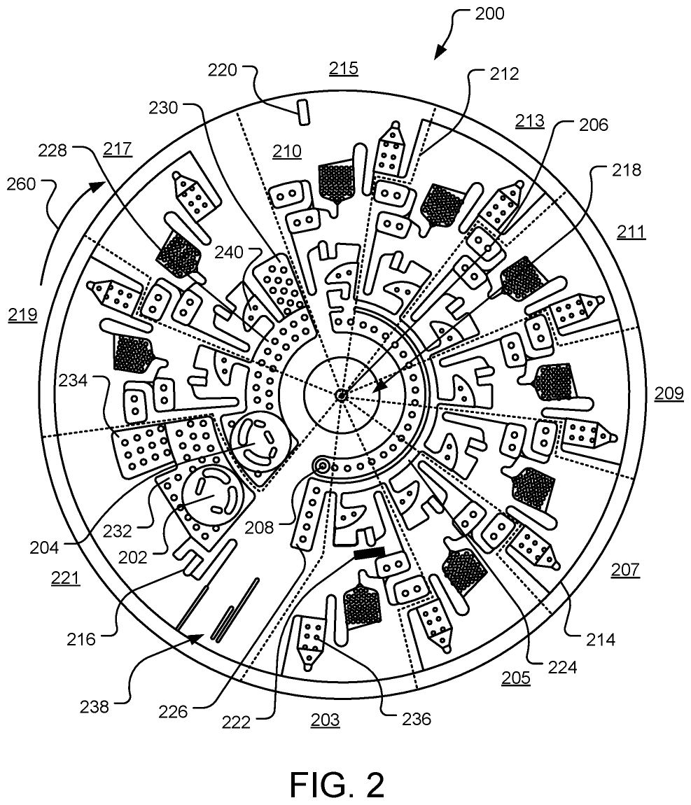

FIG. 2 is a functional schematic of an example centrifugal micro-fluidic disk 200 with an integrated reagent fluid pack 202 and an integrated control fluid pack 204 that may be used to analyze a test subject's fluid sample for detecting the presence of one or more analytes of interest referenced against integrated high and low control fluid measurements. The disk 200 comprises a substrate 210 with a center 206, at which there is a locating hole 218 for positioning the disk 200 on a motor (not shown, see e.g., motor 510 of FIG. 5) to be spun. The disk 200 spins about its center 206 as illustrated by arrow 260, which imparts a variety forces on fluids within the disk 200, including but not limited to centrifugal, Coriolis, Euler, and other forces. These forces cause fluid to move within the disk 200 in a predictable and functional manner, as discussed in further detail below.

The disk 200 includes an array of sectors 203, 205, 207, 209, 211, 213, 215, 217, 219, 221 generally arranged radially about the disk 200 and generally delineated by dotted lines (e.g., dotted line 212) in FIG. 2. Each of the sectors are assigned a specific function within the disk 200 and operate substantially independently and in parallel. For example, the disk 200 includes drug detection sectors 203, 205, 207, 209, 211, 213, 215 that are used to analyze a subject fluid sample (not shown, see e.g., subject fluid sample 710 of FIG. 7) introduced at fluid input port 208 for the presence of various drugs or other chemicals. In various implementations, each of the drug detection sectors 203, 205, 207, 209, 211, 213, 215 are used to analyze the subject fluid sample for a different drug or other chemicals thereby testing the subject fluid sample simultaneously and in parallel for a variety of drugs that may be present. While seven drug detection sectors 203, 205, 207, 209, 211, 213, 215 are illustrated on the disk 200, greater or fewer drug detection sectors may be utilized within other disks as needed for testing the subject fluid sample and as space on other disks permit.

The fluid input port 208 can have generally any shape and configuration, and fluid may enter the fluid input port 208 utilizing substantially any fluid transport mechanism, including dropping, pipetting, or pumping (e.g., via a syringe) a fluid sample into the fluid input port 208. Generally, the fluid input port 208 is in fluid communication with the drug detection sectors 203, 205, 207, 209, 211, 213, 215 in that a fluid may flow from one area to the other (e.g., from the fluid input port 208 to the drug detection sectors 203, 205, 207, 209, 211, 213, 215), either freely or using one or more transport forces and/or valves, and with or without flowing through intervening structures. The subject fluid input port 208 is connected to a subject fluid distribution zone 224 that is an arcuate input manifold commonly connected to an input of each of the drug detection sectors 203, 205, 207, 209, 211, 213, 215. The fluid distribution zone 224 is further connected to an input fluid overflow 226 that collects excess subject fluid that is not used for the drug detection sectors 203, 205, 207, 209, 211, 213, 215 and allows for an excess of subject fluid sample to be input to the fluid input port 208 without negatively affecting operation of the disk 200.

The disk 200 also includes high control sector 217 and low control sector 219 that are used to perform a similar analysis on a control sample released by the integrated control fluid pack 204 for comparison against the drug detection sectors. The control sectors 217, 219 are generally used to provide high and low points of reference, respectively, for determining relative quantities of any detected drugs or other chemicals that may be present in the subject's fluid sample as detected by any one or more of the drug detection sectors 203, 205, 207, 209, 211, 213, 215. The high and low points of reference may be used to improve an estimate of relative quantity of a drug or other chemical of interest in the subject's fluid sample (as compared to a singular control sector) as compared to a singular point of reference offered by a singular control sector. Use of a singular control sector is contemplated herein where it provides a sufficient point of reference. Further, additional control sectors may be included where some or all of the drug detection sectors 203, 205, 207, 209, 211, 213, 215 require different control measurements. In cases where detection of any quantity of a drug or other chemical of interest in the subject's fluid sample is all that is required, one of the control sectors 217, 219 or neither of the control sectors 217, 219 may be sufficient.

The control fluid pack 204 is connected to a control fluid distribution zone 228 that is commonly connected to an input of each of the control sectors 217, 219. The control fluid distribution zone 228 is further connected to a control fluid overflow 230 that collects excess control fluid that is not used for the control sectors 217, 219 and allows for an excess of control fluid sample to be input via the control fluid pack 204 without negatively affecting operation of the disk 200.

In an example implementation, the control fluid pack 204 includes a simulant of the test subject's fluid sample without the analyte of interest. The low control sector 219 is used to test the simulant and define an output that is known to be based on a sample devoid of the analyte of interest. In contrast, the high control section 217 includes a relatively large quantity of dehydrated analyte of interest in one of its chambers (e.g., a metering chamber 240). As the simulant moves through the metering chamber 240, it picks up the dehydrated analyte of interest. As a result, the output of the high control section 217 is known to be based on a sample with a particularly high concentration of the analyte of interest. These high and low points can be used as references to compare outputs of the drug detection sectors 203, 205, 207, 209, 211, 213, 215 against. This further may be used to estimate a detected concentration of the analyte of interest based on the outputs of the drug detection sectors 203, 205, 207, 209, 211, 213, 215 in comparison to the high and low points defined by the control sectors 217, 219, respectively.

The disk 200 also includes a liquid reagent sector 221 that is used to fill or rehydrate one or more dehydrated chambers found in any or all of the drug detection sectors and the control sectors using liquid reagent released by the integrated liquid reagent fluid pack 202. Once released, the liquid reagent generally flows radially outward through the liquid reagent sector 221 to a reagent distribution ring 214 positioned radially outward from the reagent fluidic circuit. The integrated liquid reagent fluid pack 202 is connected to a reagent fluid distribution zone 232 that is connected to the liquid reagent sector 221. The reagent fluid distribution zone 232 is further connected to a reagent fluid overflow 234 that collects excess reagent fluid that is not used for the liquid reagent sector 221 and allows for an excess of reagent fluid to be input via the liquid reagent fluid pack 202 without negatively affecting operation of the disk 200.

After filling the reagent distribution ring 214, the liquid reagent generally flows radially inward into the drug detection sectors 203, 205, 207, 209, 211, 213, 215 and/or the control sectors 217, 219 to fill one or more chambers found within the drug detection sectors 203, 205, 207, 209, 211, 213, 215 and/or the control sectors 217, 219. This allows empty or dehydrated chambers to be used throughout the disk 200, which makes the disk 200 easier to store, shelf-stable for longer periods of time, and generally in an inert state until rehydrated using the liquid reagent sector 221.

While the liquid reagent is initially under pressure to move radially outward from the liquid reagent fluid pack 202, as the reagent distribution ring 214 fills, liquid reagent within the reagent distribution ring 214 is under pressure to move radially inward due to Stevin's law. Specifically, the liquid reagent sector 221 includes a distribution chamber 216 that is intentionally placed at a radial position on the disk 200 equal to a maximum radial position where empty or dehydrated chambers are to be filled within the drug detection sectors 203, 205, 207, 209, 211, 213, 215 and/or the control sectors 217, 219. The distribution chamber 216 and the drug detection sectors 203, 205, 207, 209, 211, 213, 215 and/or the control sectors 217, 219 function as communicating vessels connected by the reagent distribution ring 214. As Stevin's law provides that when liquid in communicating vessels under the same pressure constraints is allowed to find equilibrium, the liquid balances out to the same level in all of the containers regardless of the shape and volume of the containers, the radial position of the distribution chamber 216 on the disk 200 defines where the liquid reagent moves radially inward to within the drug detection sectors 203, 205, 207, 209, 211, 213, 215 and/or the control sectors 217, 219 to fill or rehydrate chambers therein.

The disk 200 also includes a production alignment mark 238 and a hardware alignment mark 220. The production alignment mark 238 is present to ensure proper placement and relative alignment of the sectors 203, 205, 207, 209, 211, 213, 215, 217, 219, 221 and other features of the disk 200. The hardware alignment mark 220 ensures that a corresponding drug detection system (not shown, see e.g., drug detection system 501 of FIG. 5) is spinning the disk 200 at a desired speed and accurately tracking the sectors 203, 205, 207, 209, 211, 213, 215, 217, 219, 221 and other features of the disk 200 as the disk 200 spins within the drug detection system.

In some implementations, some or all of the drug detection sectors 203, 205, 207, 209, 211, 213, 215 and/or the control sectors 217, 219 utilize a magnet (e.g., magnet 222 within sector 203) placed adjacent a portion of the sector to magnetically pre-separate the fluid sample (subject fluid sample or control sample). This may improve the quality of subsequent operations performed on the fluid sample, as discussed in further detail below. Small support posts (e.g., support post 236) are illustrated throughout the disk 200 to provide internal support for the various chambers and other internal features of the disk 200 where needed.

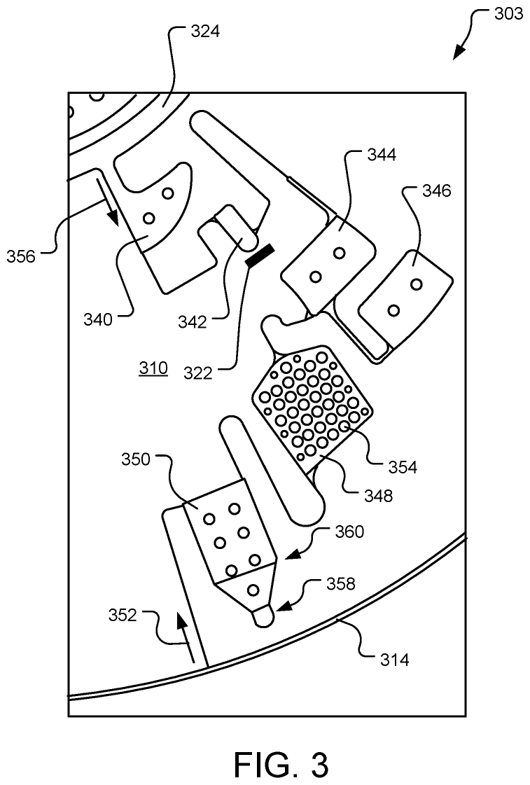

FIG. 3 is a functional schematic of a singular drug detection sector 303 within a centrifugal micro-fluidic disk (not shown, see e.g., disk 200 of FIG. 2). The disk comprises a substrate 310 with a center (not shown, see e.g., center 206 of FIG. 2). The disk spins about its center, which imparts a variety forces on fluids within the disk, including but not limited to centrifugal, Coriolis, Euler, forces. These forces cause fluid to move within drug detection sector 303 and elsewhere within the disk in a predictable and functional manner, as discussed in further detail below. The drug detection sector 303 includes a metering chamber 340, a magnetic separation chamber 342, a pair of antibody mixing chambers 344, 346, a high-density bead chamber 348, and a detection chamber 350. In various implementations, other drug detection sectors and/or control sectors within the disk may have similar chambers.

Prior to using the drug detection sector 303 for analyzing a test subject fluid sample, a portion of the drug detection sector 303 is filled or rehydrated using liquid reagent from a liquid reagent sector (not shown, see e.g., liquid reagent sector 221). The liquid reagent fills reagent distribution ring 314 and generally flows radially inward into the drug detection sector 303, as illustrated by arrow 352. The liquid reagent is used specifically to fill the detection chamber 350. The test subject fluid sample rehydrates dry high-density beads (e.g., silica bead 354) in the high-density bead chamber 348, dry antibodies in one or both of the antibody mixing chambers 344, 346, and/or dry magnetic beads found in the metering chamber 340 as it moves through these features on the disk.

The disk includes a fluid inlet port (not shown see e.g., fluid inlet port 208 of FIG. 2) that is connected to a fluid distribution zone 324 that is an arcuate input manifold connected to the drug detection sector 303. During operation, the test subject fluid sample generally flows radially outward into the drug detection sector 303, as illustrated by arrow 356. As will be described further below, a fluid, e.g., a biological fluid, such as blood, whole blood, serum, saliva, urine, or any other suitable sample matrix, can be introduced to the drug detection sector 303 via the fluid distribution zone 324. The relative sizes and proportions of the various components illustrated in FIG. 3 are simply to enable understanding of the various implementations and concepts presented herein. The respective proportions of a first feature to a second feature may be different to that illustrated.

The metering chamber 340 serves to meter the test fluid flowing into the drug detection sector 303 and mix magnetic beads with the test fluid, if applicable. The fluid continues to the magnetic separation chamber 342. In various implementations, the test fluid is a magnetically pre-separated fluid sample containing an analyte of interest (e.g., delta-9-THC). A nearby magnet 322 pulls the magnetic beads and attached undesired components (e.g., undesired metabolites) downward to a bottom of the metering chamber 340 and traps the magnetic beads and attached undesired components there. This process serves to magnetically pre-separate the test fluid, as discussed in further detail below.

In other implementations, the metering chamber 340 contains pre-separation beads that may or may not be magnetic but are significantly denser than the analyte of interest. Centrifugal forces serve to pre-separate the test fluid by pulling the pre-separation beads along with attached undesired components (e.g., undesired metabolites) downward to the bottom of the separation chamber 342.

The pre-separated test fluid continues to the antibody mixing chambers 344, 346 where antibodies are mixed with the test fluid. The test fluid with antibodies continues to the high-density bead chamber 348 where high-density beads are mixed in. The test fluid with antibodies and high-density beads continues to the detection chamber 350.

The detection chamber 350 can comprise a first region 358 and a second region 360. The first region 358 collects the test fluid at a bottom of the detection chamber 350. The first region 358 is a testing area, e.g., is utilized to test the fluid sample for a particular compound of interest. The second region 360 collects a portion of the fluid sample with a lower density than the test fluid that collects in the first (or test) region 358, wherein the difference in fluid densities can be utilized to separate one or more test fluid components, e.g., separate first silica beads having a first density from second silica beads having a second density, wherein the first density is higher than the second density, as further described.

As further described below, the disk can be rotated, thereby generating a centrifugal force that acts on one or more components (e.g., the test sample, first beads, second beads, etc.) within the drug detection sector 303 to facilitate separation of a component (e.g., a compound of interest) from a plurality of other components that are also located in the test sample. More specifically, centrifugal separation can be utilized to separate delta-9-THC compound(s) from a plurality of metabolites formed either from a previously existing delta-9-THC compound or another cannabinoid. In another example, an antibody which has bound to an analyte can be separated from a sample to enable determination of whether an individual is using a restricted drug (e.g., a class 1 drug).

While a single drug detection sector 303 is illustrated in FIG. 3, any number of sectors can be present on a disk (e.g., an array of sectors may be arranged extended generally radially from a center of the disk) to facilitate a plurality of tests to be conducted at the same time. As the disk is rotated, a centrifugal force is generated. The centrifugal force may serve to separate higher-density components from lower-density components of the test fluids.

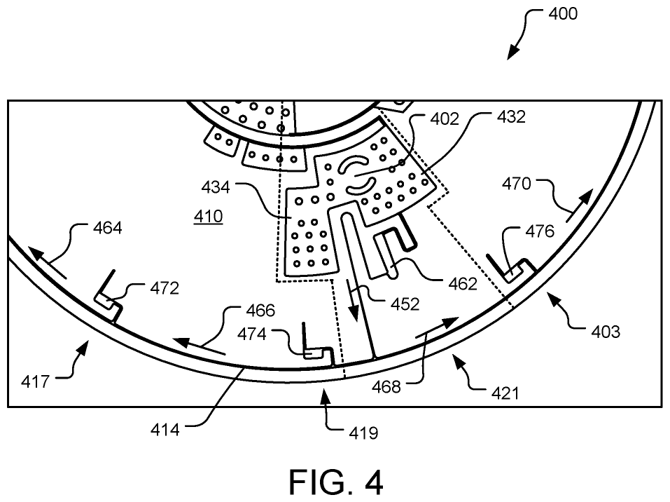

FIG. 4 is a functional schematic of an integrated reagent sector 421 within a centrifugal micro-fluidic disk 400. The disk 400 comprises a substrate 410 with a center (not shown, see e.g., center 206 of FIG. 2). The disk 400 spins about its center, which imparts a variety forces on fluids within the disk 400, including but not limited to centrifugal, Coriolis, Euler, forces. These forces cause fluid to move within the integrated reagent sector 421 and elsewhere within the disk 400 in a predictable and functional manner, as discussed in further detail below. The integrated reagent sector 421 includes an integrated liquid reagent fluid pack 402, a reagent fluid distribution zone 432, a reagent overflow zone 434, and a metering chamber 462.

The integrated reagent sector 421 is used to fill or rehydrate one or more dehydrated chambers found in any or all of drug detection sectors (e.g., drug detection sector 403, illustrated in part) and control sectors (e.g., control sectors 417, 419, illustrated in part) on the disk 400 using liquid reagent released by the integrated liquid reagent fluid pack 402. Once released, the liquid reagent generally flows radially outward through the liquid reagent sector 421 to a reagent distribution ring 414 positioned radially outward from the reagent fluidic sector 421, as illustrated by arrow 452. The integrated liquid reagent fluid pack 402 is connected to the reagent fluid distribution zone 432 that is connected to the liquid reagent sector 421. The reagent fluid distribution zone 432 is further connected to a reagent overflow zone 434 that collects excess reagent fluid that is not used for the liquid reagent sector 421 and allows for an excess of reagent fluid to be input via the liquid reagent fluid pack 402 without negatively affecting operation of the disk 400.

After filling the reagent distribution ring 414, as illustrated by arrows 464, 466, 468, 470, the liquid reagent generally flows radially inward into the drug detection sectors and/or the control sectors to fill or rehydrate dehydrated chambers found within the applicable sectors, specifically detection chambers 472, 474, 476. In other implementation, the detection chambers 472, 474, 476 contain dehydrated density media and the liquid reagent fluid pack 402 merely contains a buffer fluid that is used to rehydrate the dehydrated density media within the detection chambers 472, 474, 476. Either way, this allows empty or dehydrated chambers to be used throughout the disk 400, which make the disk 400 easier to store, shelf-stable for longer periods of time, and generally in an inert state until rehydrated using the liquid reagent sector 421.

The reagent fluid can be formed from any suitable material of higher mean density. For example, the reagent fluid can be a colloidal suspension of silicon nanoparticles (e.g., PERCOLL) with a density of about 1.4-1.6 g/cm³. Surfactants and salts can be added to stabilize any biological reactions (e.g., with 0.05% (weight/volume) Tween-20, 0.15M sodium chloride, 0.02M sodium phosphate, pH 7.4-8.5). Further, the reagent fluid may include one or more of dextran, polyethylene glycol, glycerol, sorbitol, iodixanol, cesium chloride, perfluoro decalin, etc., stored on the disk 400 in liquid or solid (to be rehydrated) form.

FIG. 5 is a schematic of a centrifugal micro-fluidic disk drug detection system 501 used to determine if a fluid sample includes a drug-related compound. The drug detection system 501 receives a disk 500 including an array of test sectors (e.g., test sector 520). A motor 510 is coupled to the disk 500 and spins the disk 500, generating centrifugal force(s). A detection module 519 is positioned to detect (or determine an absence of) a signal generated by, for example, a labeled antibody located in the each of the test sectors (e.g., within test region 358 of FIG. 3).

A plurality of detection techniques can be utilized for the various embodiments presented herein. For example, an optical detection technique (e.g., fluorescence, luminescence, absorbance, time-resolved fluorescence, refractive index monitoring, etc.); an electrochemical technique; an amperometric technique, etc. An antibody may be labeled or tagged (e.g., to fluoresce) when stimulated by light during a detection process, thereby enabling a location of the antibody to be determined, and accordingly, whether a drug-related is present (or not). It is to be appreciated that any suitable tag can be utilized, for example, organic dyes (e.g., cyanine dye, Alexa Fluor (Invitrogen™), fluorescein, fluorescein isothiocyanate, etc.), inorganic quantum dots (e.g., cadmium selenide quantum dots), polystyrene nanoparticles or other particles of a similar makeup (e.g., FLUOSPHERES), organometallic dyes (e.g., europium-based dyes), etc.

In some implementations, the lack of presence of the fluorescing antibody at a particular location can be an indication that a drug-related compound is present in a fluid sample. The detection module 519 can include an emitter 521 and a sensor 523. In an example antibody that is configured to fluoresce, the emitter 521 can be a light source configured to transmit light 522 in accordance with a stimulation frequency of the fluorescent antibody. The sensor 523 can detect light 524 transmitted (emitted, reflected, and/or generated) from the antibody as a function of the fluorescence process. More specifically, the emitter 521 may comprise a laser and the sensor 523 may comprise optics suitable for optical detection of fluorescence from fluorescent labels (e.g., as applied to an antibody). In another example, other detectors, such as electronic detectors, may be used. Generally, the emitter 521 and the sensor 523 may be selected based upon the tagging system selected for the antibody or other ligand.

In some implementations, a plurality of emitters 521 and a plurality of sensors 523 can be included in the system 501, wherein the emitters 521 and sensors 523 are selected for use based upon the type of drug being tested for, a detection labeling of a component under test, e.g., a labeled antibody, etc. By utilizing a plurality of co-located emitters 521 and sensors 523, a single system 501 can be utilized to test for a plurality of different drug-related compounds. In other implementation, a singular emitter 521 and sensor 523 can be used for all sectors on the disk 500, while the fluorophores and/or reagents used in the various sectors may vary.

The drug detection system 501 includes a control system 530 that controls operation of the drug detection system 501. The control system 530 includes a processor 535 and a memory 536. The memory 536 includes software components that are executable by the processor 535.

An identification component 540 may obtain and store identification data regarding an identification of an individual who is undergoing testing. The identification component 540 may receive information from a data input 545 (e.g., a card reader that reads identification information from an identification card 546, such as a driver's license). Further, the data input 545 can further include an input device such as a keypad and display, wherein information is entered to complete data fields, respond to questions and data prompts, etc. Data collected by the input component 545 can be stored in a data store 547. The identification data may be utilized to uniquely mark the disk 500, e.g., for subsequent analysis. For example, a first sector on the disk 500 may be utilized to conduct a drug test on a magnetically pre-separated fluid sample, while a second sector may be utilized to store a second magnetically pre-separated fluid sample that can be utilized for subsequent testing. More specifically, if a roadside test conducted with the drug detection system 501 generates a positive result, an individual may be operating a vehicle while under the influence of an illegal drug. A stored magnetically pre-separated fluid sample can be utilized to conduct subsequent testing, for example, at a testing center having equipment available that has a higher degree of specificity and/or accuracy to determine a drug in a magnetically pre-separated fluid sample.

A reader/writer component 550 can be utilized to read identifying information from the disk 500 or write information (e.g., as obtained from the input component 545) to the disk 500 to enable unique identification of the disk 500. For example, the disk 500 may have a writeable region (e.g., similar to a writeable region on a writeable compact disk). Further, the reader/writer component 550 may include a printing head to print data onto a surface of the disk 500. Further, the reader/writer component 550 can be a laser device that can burn data onto a surface of the disk 500. Still further, the reader/writer component 550 can be a QR, barcode, or other disk information reader.

A detection component 560 is further included in the memory 536 that configures operation of the system 501 to detect presence of a tagged compound. For example, an instruction input may be received (e.g., via input 545) regarding a particular type of drug that is to be tested for, and the detection component 560 selects the emitter 521 and sensor 523 to assay for the tagged analyte associated with the instructed drug to be detected. Further, the detection component 560 can provide instructions to the person operating the system 501 (e.g., a police officer) to assist the person in correctly performing the test.

The detection component 560 may also coordinate operation of the various components and features of the system 501. For example, the detection component 560 may initiate and direct processing of the disk 500 with the system 501. Once the disk 500 is located in the system 501, e.g., the disk 500 is located on the motor 510, a timer (not shown) can be initiated to control a duration of incubation of a fluid sample within the disk 500. Upon completion of the incubation period, the fluid sample can undergo testing and yield results generated therefrom, etc. The various operations required to initiate a test through to test completion can be controlled by the detection component 560.

An output component 570 may generate a test result 575 and transmit the test result 575 to an external system. Further, a display 580 (e.g., a touchscreen) can be further included in the system 501 to facilitate display of results, presentation of interactive screens, receive input information, etc. A battery 590 can be further included in the system 501 to enable power to, and operation of one or more components included in the system 501 (e.g., the control system 530, the motor 510, the detector module 519, etc.). All or selected components illustrated in FIG. 5 may be housed in a common housing. Disks may be selectively placed on and removed from the motor 510, such that multiple disks may be analyzed by the system 501 in series. The motor 510 can be a centrifugal and/or stepper motor. The motor 510 can be positioned relative to the detection module 519 such that, when the disk 500 is situated on the motor 510, the disk 500 is positioned such that a detection region is aligned with the detection module 519.

In another example, although not explicitly shown in FIG. 5, one or more actuators can be coupled to the motor 510 and/or disk 500 such that the disk 500 is moved relative to the detection module 519 responsive to signals from the processor 535. The processor 535 can provide a control signal(s) to the motor 510 to rotate the disk 500 at a selected speed(s) for a selected time(s). The processor 535 can provide a control signal(s) to the detection module 519, wherein the control signal controls which emitter 521, sensor 523, etc., are to be used.

A separate computing system (e.g., network server) may also be used to implement some of the functional aspects of the control system 530 or add additional functional aspects. The computer system may be capable of executing a computer program product embodied in a tangible computer-readable storage medium to execute a computer process. Data and program files may be input to the computer system, which reads the files and executes the programs therein using one or more processors. Some of the elements of the computer system may include an I/O section, a central processing unit (e.g., processor), and a program memory. There may be one or more processors, such that the processor of the computer system comprises a single central processing unit, or a plurality of processing units, commonly referred to as a parallel processing environment. The computer system may be a conventional computer, a distributed computer, or any other type of computer. The described technology is optionally implemented in software loaded in memory stored on a storage unit, and/or communicated via a wired or wireless network link on a carrier signal, thereby transforming the computer system to a special purpose machine for implementing the described operations.

A communication interface may be capable of connecting the control system 530 to a wireless network, through which the drug detection system 501 can receive instructions and data embodied in a carrier wave. When used in a local-area-networking (LAN) environment, the computer system is connected (by wired connection or wirelessly) to a local network through the network interface or adapter, which is one type of communications device. When used in a wide-area-networking (WAN) environment, the computer system typically includes a modem, a network adapter, or any other type of communications device for establishing communications over the wide area network. In a networked environment, program modules depicted relative to the computer system or portions thereof, may be stored in a remote memory storage device. It is appreciated that the network connections described are exemplary and other means of and communications devices for establishing a communications link between the drug detection system 501, the control system 530, and the wireless network may be used.

In an example implementation, a user interface software module and other modules may be embodied by instructions stored in memory (e.g., memory 536) and/or a storage unit and executed by a processor (e.g., processor 535). Further, local computing systems, remote data sources and/or services, and other associated logic represent firmware, hardware, and/or software may be configured to assist in obtaining breath alcohol content measurements. The delta-9-THC detection sequences disclosed herein may be implemented using a general-purpose computer and specialized software (such as a server executing service software), a special purpose computing system and specialized software (such as a mobile device or network appliance executing service software), or other computing configurations. In addition, delta-9-THC measurements and computations may be stored in the memory 536 and executed by the processor 535.

The magnetic pre-separation sequence and/or delta-9-THC detection sequences disclosed herein may be implemented in software executing on a stand-alone computer system, whether connected to the drug detection system 501 or not. In yet another implementation, the magnetic pre-separation sequence and/or delta-9-THC detection sequences disclosed herein may be integrated into the drug detection system 501 or one or more other devices.

Data storage and/or memory may be embodied by various types of storage, such as hard disk media, a storage array containing multiple storage devices, optical media, solid-state drive technology, read-only memory (ROM), random access memory (RAM), and other technology. The operations may be implemented in firmware, software, hard-wired circuitry, gate array technology and other technologies, whether executed or assisted by a microprocessor, a microprocessor core, a microcontroller, special purpose circuitry, or other processing technologies.

For purposes of this description and meaning of the claims, the terms “computer readable storage media” and “memory” refer to a tangible (or non-transitory) data storage device, including non-volatile memories (such as flash memory, disc drives, and the like) and volatile memories (such as dynamic random-access memory and the like). The computer instructions either permanently or temporarily reside in the memory, along with other information such as data, virtual mappings, operating systems, applications, and the like that are accessed by a computer processor to perform the desired functionality. The terms “computer readable storage media” and “memory” expressly do not include a transitory medium such as a carrier signal, but the computer instructions can be transferred to the memory wirelessly.

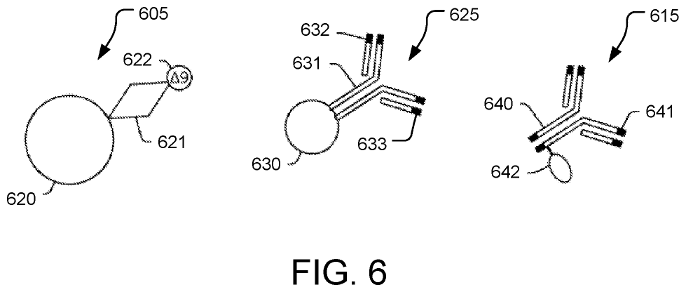

FIG. 6 illustrates example additives 605, 625, 615 to aid detection of delta-9-THC in a fluid sample. The first additive 605 includes a high-density bead 620 with a first protein 621 attached thereto, wherein the first protein 621 is delta-9-THC specific and has a delta-9-THC molecule 622 is attached to the first bead 620 via the first protein 621. The second additive 625 includes a magnetic bead 630 with an antibody 631 attached thereto, wherein the antibody 631 is specific to (e.g., binds to) metabolites (e.g., delta-9-THC metabolites and other metabolites) via the regions 632, 633. In various implementations, the antibody 631 may be any form of ligand, including but not limited to antibodies, fragment antigen-binding (FAB) fragments, aptamers, etc. that can bind a target of interest, such as delta-9-THC metabolites. The third additive 615 is an antibody 640 with regions 641 specific to a delta-9-THC molecule, and further has a fluorophore 642 attached thereto. As further described, detecting the presence of the third additive 615 by its fluorophore 642 in test region 358 of FIG. 3 (e.g., based upon its attachment to the first additive 605) enables a determination of whether a fluid sample includes delta-9-THC.

The beads 620, 630 can be selected from a variety of suitable materials (e.g., inert materials with respect to reacting with delta-9-THC molecule(s) and metabolites thereof). Example materials for the beads 620 include silica (and associated materials, e.g., glass, hollow glass spheres, etc.) and other materials that have a density greater than that of surrounding fluid in the regions 358, 360 of FIG. 3. This allows centrifugal separation to pull the beads 620 into the test region 358 of FIG. 3 for testing. In an example implementation, the first beads 620 can be formed from silica having a density of about 1.8-2.0 g/cm³.

Example materials for the beads 630 include magnetic microspheres (MMs), such as ferromagnetic particles (e.g., magnetite (Fe₃O₄) or magnetite (gamma Fe₂O₃) coated with polymers, silica, or hydroxyl apatite) or superparamagnetic particles such as polystyrene particles compounded with metal nanoparticles, metals such as gold, silver, iron, polyamide, melamine, etc. While the first beads 620 are illustrated as larger than the second beads 630, this distinction is purely for the purposes of illustration and understanding. The first beads 620 and the second beads 630 can be of the same size, or of a different size (e.g., the first beads 620 can be smaller than the second beads 630). The first beads 620 and the second beads 630 can each have a size ranging from about 0.1 micrometers to about 50 micrometers, for example. In further implementations, the first beads 620 and/or the second beads 630 are nanoparticles, each having a size ranging from about 0.001 micrometers to about 0.1 micrometers. In further iterations, the first beads 620 and the second beads 630 could be of the same material and size.

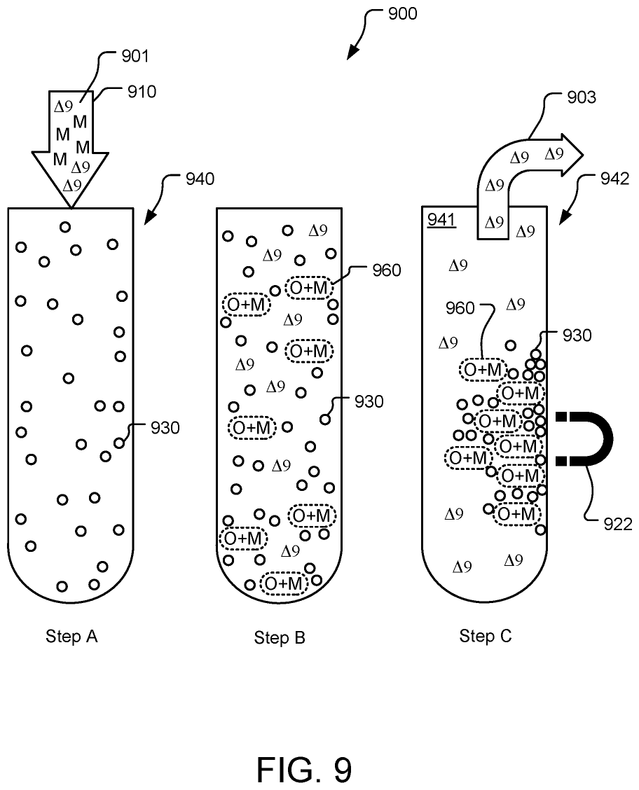

As previously mentioned, habitual users of marijuana will have high levels of various metabolites in their system. In marijuana, delta-9-THC is the active ingredient capable of causing intoxication and therefore the target of interest for screening for intoxication. The second beads 630 are used to attach to various metabolites (e.g., delta-9-THC metabolites) in a fluid sample and then the magnetic properties of the beads 630 are used to separate the metabolites from other components of the fluid sample in a magnetic pre-separation sequence of a fluid sample assay (see e.g., sequences 700, 900 of FIGS. 7 and 9, respectively), as discussed in further detail below.

The first beads 620 have a high-density that can be utilized to bind fluorophore 642, that in turn is used to separate delta-9-THC compounds from other components of the fluid sample. Further, the respective concentration of first beads 620 to second beads 630 can be tuned such that there are sufficient metabolite-specific binding sites to effectively deplete the metabolites but not so much that potential cross-reactivity with delta-9-THC depletes a desired signal (e.g., during concentration measurement and determination by the detection module 519 of FIG. 5). In other implementations, the second beads 630 are used to deplete other unwanted components beyond metabolites.

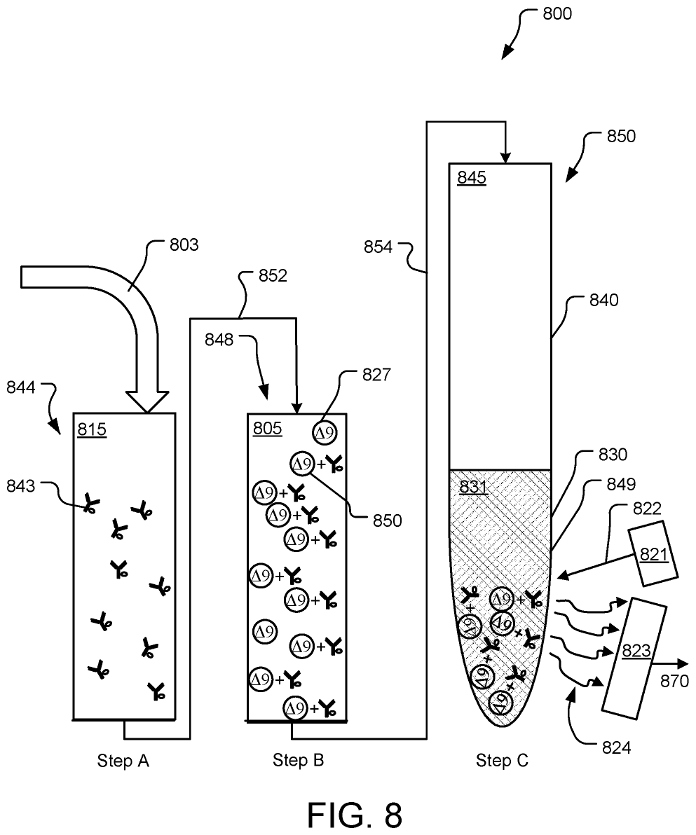

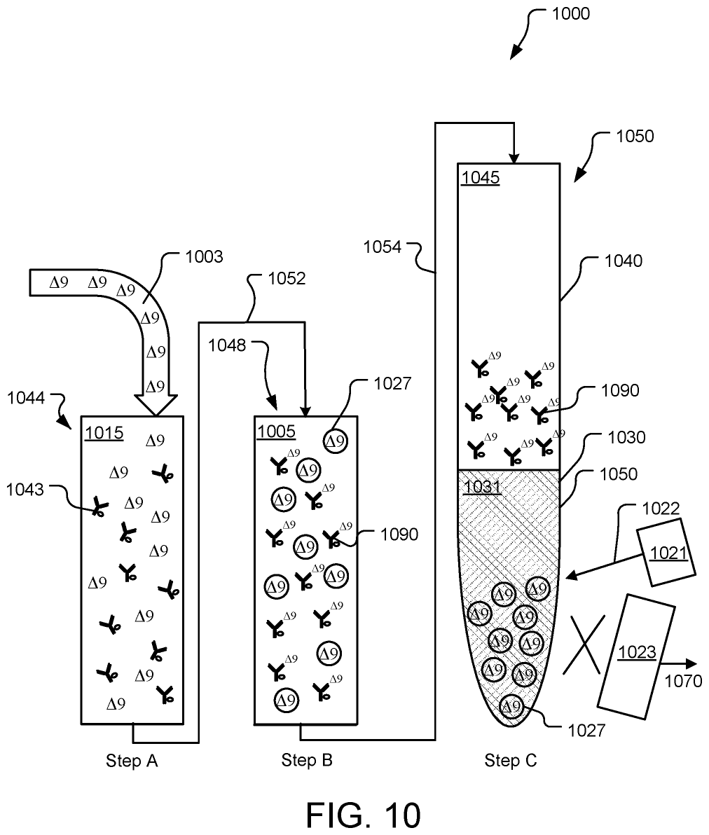

In FIGS. 7-10, sequences 700, 800, 900, 1000 illustrate an implementation for detecting the presence of a drug and/or its metabolites. In sequences 700, 800 of FIGS. 7, 8, respectively, an example scenario is illustrated where a sample does not contain delta-9-THC (e.g., the volume of delta-9-THC present is below a legal limit). In sequences 900, 1000 of FIGS. 9, 10, respectively, an example scenario is illustrated where a sample contains delta-9-THC (e.g., the volume of delta-9-THC present is above a legal limit).

FIG. 7 illustrates a magnetic pre-separation sequence 700 separating metabolites (M) from a fluid sample 710 including various metabolites (M) but no delta-9-THC. The fluid sample 710 (e.g., a saliva sample collected from an individual to be tested) is collected within a drool cup or other container (not shown) and may be mixed with an extraction buffer 701 and then sealed. The extraction buffer 701 suspends and distributes the metabolites (M) within the fluid sample 710. The fluid sample 710 may range from 0.01-1.0 ml and is from an individual who has not recently consumed marijuana and hence has only metabolites (M) in their saliva.

Metering chamber 740 illustrated in Step A is filled with assay additive 625 of FIG. 6, including magnetic beads (e.g., magnetic bead 730) alone within the metering chamber 740 or suspended in a buffer. In implementations where the magnetic beads are stored within the metering chamber 740 in a dehydrated state, the fluid sample 710 re-hydrates and suspends the assay additive 625 of FIG. 6, including the magnetic beads, within the extraction buffer 701. A measured quantity of the fluid sample 710 is allowed to pass through the metering chamber 740 in Step A. The disk includes a fluid inlet port (not shown see e.g., fluid inlet port 208 of FIG. 2) that is fluidly connected to the disk metering chamber 740. During operation, the test subject fluid sample generally flows radially outward from the fluid inlet port into the metering chamber 740.

Between Steps A and B, the fluid sample 710 and the assay additive 625 of FIG. 6, including the magnetic beads, are mixed and an incubation period of time is allowed to pass. During this time, the magnetic beads with antibodies 631 of FIG. 6 attached attract metabolites (e.g., carboxy (COOH), hydroxy (OH)). Delta-9-THC, if it were present, would remain unattached to the magnetic beads. Sufficient magnetic beads are included in the metering chamber 740 to bond with substantially all the metabolites (M) from the fluid sample 710 added to the metering chamber 740, thereby forming magnetic bead combinations (e.g., magnetic bead combination 760). Further, between Steps A and C, the combined fluid sample 710 and assay additive 625 of FIG. 6, including the magnetic beads, moves from the metering chamber 740 to a magnetic separation chamber 742 within the disk.

In Step C, a magnet 722 is placed adjacent to the magnetic separation chamber 742 and generally at its side or bottom, away from an outlet of the fluid. While illustrated as a horseshoe magnet, the magnet 722 may be any size, shape, or configuration and may be placed adjacent the exterior of any portion of the magnetic separation chamber 742. The magnet 722 attracts the magnetic beads with the metabolites (M) from the fluid sample 710 now attached (magnetic bead combinations) as provided in Step B. As a result, the “O+M” and “O” components that include the magnetic beads 630 are drawn to the magnet 722 and cluster around the magnet 722, while the remaining components of the fluid 741 (including Delta-9-THC, if it were present), remains substantially evenly suspended within the fluid 741. As the metabolites (M) are unwanted components for a further delta-9-THC detection sequence (see e.g., delta-9-THC detection sequence 800 of FIG. 8), the metabolites (M) are substantially separated from the fluid 741 in Step C of the magnetic pre-separation sequence 700.

A detection sample 703 of the magnetically pre-separated fluid 741 is pulled out of the magnetic separation chamber 742. As the fluid sample 710 includes various metabolites (M), but no delta-9-THC, and the metabolites (M) are bonded to the magnetic beads and magnetically separated from the remainder of the fluid 741, the sample 703 contains neither of delta-9-THC nor various metabolites (M) thereof. The detection sample 703 effectively excludes the metabolites (M) and/or other unwanted substances prior to the following delta-9-THC detection sequence 800 of FIG. 8. In various implementations, this may improve the accuracy and repeatability of the delta-9-THC detection sequence 800 of FIG. 8. The detection sample 703 may range from 0.01-10 ml and is partially comprised of a buffer fluid.

FIG. 8 illustrates a delta-9-THC detection sequence 800 using fluorescence within a testing area 830 of a detection chamber 849 within a micro-fluidic disk (not shown, see e.g., disk 100 of FIG. 1) with the magnetically pre-separated detection sample 703 of FIG. 7 (here illustrated as magnetically pre-separated detection sample 803). In Step A, the detection sample 803 is added to mixing chamber(s) 844 that includes an additive 815 including antibodies (e.g., antibody 843), the antibodies each having regions specific to a delta-9-THC molecule (see also third additive 615 of FIG. 6) and a fluorophore attached thereto. The detection sample 803 and the additive 815 are mixed in the mixing chamber(s) 844 and allowed to incubate for a period of time (e.g., 1-60 minutes). During the Step A incubation period, the antibodies within the additive 815 bond with any delta-9-THC, if it were present within the detection sample 803 (see e.g., Step A of FIG. 10).

In some implementations, the additive 815 including antibodies is stored within the mixing chamber(s) 844 in a dehydrated state. The magnetically pre-separated detection sample 803 rehydrates and suspends the additive 815 including antibodies therein. A measured quantity of the mixed additive 815 and magnetically pre-separated detection sample 803 is allowed to pass through the mixing chamber(s) 844 in Step A and enter high-density beads mixing chamber 848 in Step B, as illustrated by arrow 852.

In Step B, another additive 805 including high-density beads (e.g., high-density bead 827) each with a first protein that is delta-9-THC specific and with a delta-9-THC simulant molecule attached thereto (see also first additive 605 of FIG. 6) is included within the high-density beads mixing chamber 848. The incoming mixed additive 815 and magnetically pre-separated detection sample 803 is mixed with the additive 805 including high-density beads and allowed to incubate for another period of time (e.g., 1-60 minutes). During the Step B incubation period, in the absence of actual delta-9-THC from the detection sample 803, the antibodies from the additive 815 bond with the high-density beads with the delta-9-THC simulant from the additive 805 forming high-density bead combinations (e.g., high-density bead combination 850).

In some implementations, the additive 805 including high-density beads is stored within the high-density beads mixing chamber 848 in a dehydrated state. The incoming mixed additive 815 and magnetically pre-separated detection sample 803 rehydrates and suspends the additive 805 including high-density beads therein. A measured quantity of the additives 805, 815, and the magnetically pre-separated detection sample 803 is allowed to pass through the high-density beads mixing chamber 848 in Step B and enter detection chamber 849 in Step C, as illustrated by arrow 854.

The detection chamber 849 includes at least a first region (or testing area) 830 and a second region (or input area) 840. Other detection chambers may have additional regions for further separation and detection. The first region 830 includes a first fluid 831 closest to a closed end and furthest from an input end of the detection chamber 849. The first region 830 is a testing area in that it is utilized to assay a sample for a particular compound of interest (see e.g., Step C, discussed below). The second region 840 includes a second buffer fluid 845 closest to the input end and furthest from the closed end of the detection chamber 849. The first fluid 831 has a higher density than the second fluid 845, wherein the difference in fluid densities can be utilized to separate out one or more components of interest, as detailed below in Step C.

At Step C, the detection chamber 849 and contents therein is rotated by a motor (see e.g., motor 510 of FIG. 5), thereby subjecting the detection chamber 849 and its contents to a centrifugal force. Application of the centrifugal force causes the high-density bead combinations to move radially outward. Owing to the density of the high-density beads being greater than the densities of buffer fluids 831, 845, the high-density beads (and attached antibodies with fluorophores attached thereto) enter and cluster toward the bottom of the testing area 830 in the closed end of the detection chamber 849.

A light source 821, which can be located outside of but proximate to the testing area 830 emits light 822 directed towards the high-density bead combinations. When stimulated by electromagnetic energy (e.g., light) the fluorophores attached to the high-density bead combinations will fluoresce. Emitted light 824 generated by the fluorescence effect is captured and/or detected by a detector 823. The detector 823 may generate a signal 870 indicating a magnitude of fluorescence measured by the detector 823, or whether any fluorescence was measured (e.g., a simple binary signal, 1=fluorescence detected, 0=no fluorescence detected), or a more granular measurement of fluorescence.

A magnitude of the signal 870 may be used to determine that a high proportion (e.g., a majority) of fluorophore-labelled antibodies originally in the third additive 815 are now located in the testing area 830, and accordingly, the delta-9-THC specific antibodies were only able to attach to the delta-9-THC simulant molecules attached to the high-density beads. Hence, a conclusion may be drawn that the magnetically pre-separated detection sample 803 (and original fluid sample 710 of FIG. 7) had no delta-9-THC molecules present, or a level of delta-9-THC molecules present that is below a given legal limit.

FIG. 9 illustrates magnetic pre-separation sequence 900 separating metabolites (M) from a fluid sample 910 including various metabolites (M) and delta-9-THC (Δ9). The fluid sample 910 (e.g., a saliva sample collected from an individual to be tested) is collected within a drool cup or other container (not shown) or swab and may be mixed with an extraction buffer 901 and then sealed. The extraction buffer 901 suspends and distributes the metabolites (M) and the delta-9-THC (Δ9) within the fluid sample 910. The fluid sample 910 may range from 0.1-10.0 ml and is from an individual who has recently consumed marijuana and hence has delta-9-THC (Δ9) and various metabolites (M) thereof in their saliva.

Metering chamber 940 illustrated in Step A is filled with assay additive 625 of FIG. 6, including magnetic beads (e.g., magnetic bead 930) alone within the metering chamber 940 or suspended in a buffer. In implementations where the magnetic beads are stored within the metering chamber 940 in a dehydrated state, the fluid sample 910 rehydrates and suspends the assay additive 625 of FIG. 6, including the magnetic beads, within the extraction buffer 901. A measured quantity of the fluid sample 910 is allowed to pass through the metering chamber 940 in Step A. The disk includes a fluid inlet port (not shown see e.g., fluid inlet port 208 of FIG. 2) that is fluidly connected to the metering chamber 940. During operation, the test subject fluid sample generally flows radially outward from the fluid inlet port into the metering chamber 940.

Between Steps A and B, the fluid sample 910 and the assay additive 625 of FIG. 6, including magnetic beads are mixed and an incubation period of time is allowed to pass. During this time, the magnetic beads with antibodies 631 of FIG. 6 attached attract metabolites (e.g., carboxy (CH), hydroxy (OH)). The delta-9-THC (Δ9) remains unattached to the magnetic beads. Sufficient magnetic beads are included in the metering chamber 940 to bond with substantially all the metabolites (M) from the fluid sample 910 added to the metering chamber 940, thereby forming magnetic bead combinations (e.g., magnetic bead combination 960). Further, between Steps A and C, the combined fluid sample 910 and assay additive 625 of FIG. 6, including the magnetic beads, moves from the metering chamber 940 to a magnetic separation chamber 942 within the disk.

In Step C, a magnet 922 is placed adjacent to the magnetic separation chamber 942 and generally at its side or bottom, away from an outlet of the fluid. The magnet 922 attracts the magnetic beads with the metabolites (M) from the fluid sample 910 now attached (magnetic bead combinations) as provided in Step B. As a result, the “O+M” and “O” components that include the magnetic beads are drawn to and cluster around the magnet 922, while the remaining components of the fluid 941 (including delta-9-THC (Δ9)), remains substantially evenly suspended within the fluid 941. As the metabolites (M) are unwanted components for a further delta-9-THC detection sequence (see e.g., delta-9-THC detection sequence 1000 of FIG. 10), the metabolites (M) are substantially separated from the fluid 941 in Step C of the magnetic pre-separation sequence 900.

A detection sample 903 of the magnetically pre-separated fluid 941 is pulled out of the magnetic separation chamber 942. As the fluid sample 910 included delta-9-THC (Δ9) and various metabolites (M) thereof, but the metabolites (M) are bonded to the magnetic beads and magnetically separated from the remainder of the fluid 941, the sample 903 contains the delta-9-THC (Δ9) but not the various metabolites (M) thereof. The detection sample 903 effectively excludes the metabolites (M) and/or other unwanted substances prior to the following delta-9-THC detection sequence 1000 of FIG. 10. In various implementations, this may improve the accuracy and repeatability of the delta-9-THC detection sequence 1000 of FIG. 10. The detection sample 903 may range from 0.01-10 ml and is partially comprised of a buffer fluid with a relatively even distribution of the delta-9-THC (Δ9).

FIG. 10 illustrates a delta-9-THC detection sequence 1000 using fluorescence within a testing area 1030 of a detection chamber 1050 within a micro-fluidic disk (not shown, see e.g., disk 100 of FIG. 1) with the magnetically pre-separated detection sample 903 of FIG. 9 (here illustrated as magnetically pre-separated detection sample 1003). In Step A, the detection sample 1003 is added to mixing chamber(s) 1044 that includes an additive 1015 including antibodies (e.g., antibody 1043), the antibodies each having regions specific to a delta-9-THC molecule (see also third additive 615 of FIG. 6) and a fluorophore attached thereto. The detection sample 1003 and the additive 1015 are mixed in the mixing chamber(s) 1044 and allowed to incubate for a period of time (e.g., 1-60 minutes). During an incubation period between Step A and Step B, the antibodies within the additive 1015 bond with the delta-9-THC (Δ9) molecules present within the detection sample 1003 to form antibody combinations (e.g., antibody combination 1090).

In some implementations, the additive 1015 including antibodies is stored within the mixing chamber(s) 1044 in a dehydrated state. The magnetically pre-separated detection sample 1003 rehydrates and suspends the additive 1015 including antibodies therein. A measured quantity of the mixed additive 1015 and magnetically pre-separated detection sample 1003 is allowed to pass through the mixing chamber(s) 1044 in Step A and enter high-density beads mixing chamber 1048 in Step B, as illustrated by arrow 1052.

In Step B, another additive 1005 including high-density beads (e.g., high-density bead 1027) each with a first protein that is delta-9-THC specific and with a delta-9-THC simulant molecule attached thereto (see also first additive 605 of FIG. 6) is included within the high-density beads mixing chamber 1048. The incoming mixed additive 1015 and magnetically pre-separated detection sample 1003 is mixed with additive 1005 including high-density beads and allowed to incubate for another period of time (e.g., 1-60 minutes). During the Step B incubation period, in the absence of actual delta-9-THC from the detection sample 1003, the antibodies from the additive 1015 would bond with the high-density beads with the delta-9-THC simulant from the additive 1005. However, owing to the Δ9 molecules being already attached to the antibodies within the additive 1015, there are no (or a minimal number of) delta-9-THC specific antibodies remaining to attach to the high-density beads. Hence, there are few or none of the antibody fluorophores attached to the high-density beads and the high-density beads generally remain unattached to other molecules within the additive 1005.

In some implementations, the additive 1005 including high-density beads is stored within the high-density beads mixing chamber 1048 in a dehydrated state. The incoming mixed additive 1015 and magnetically pre-separated detection sample 1003 rehydrates and suspends the additive 1005 including high-density beads therein. A measured quantity of the additives 1005, 1015 and the magnetically pre-separated detection sample 1003 is allowed to pass through the high-density beads mixing chamber 1048 in Step B and enter detection chamber 1050 in Step C, as illustrated by arrow 1054.

The detection chamber 1050 includes at least a first region (or testing area) 1030 and a second region (or input area) 1040. Other detection chambers may have additional regions for further separation and detection. The first region 1030 includes a first fluid 1031 closest to a closed end and furthest from an input end of the detection chamber 1050. The first region 1030 is a testing area in that it is utilized to assay a sample for a particular compound of interest (see e.g., Step C, discussed below). The second region 1040 includes a second buffer fluid 1045 closest to the input end and furthest from the closed end of the detection chamber 1050. The first fluid 1031 has a higher density than the second fluid 1045, wherein the difference in fluid densities can be utilized to separate out one or more components of interest, as detailed below in Step C.

At Step C, the detection chamber 1050 and contents therein is rotated by a motor (see e.g., motor 510 of FIG. 5), thereby subjecting the detection chamber 1050 and its contents to a centrifugal force. Application of the centrifugal force causes the high-density bead combinations to move radially outward. Owing to the density of the high-density beads being greater than the densities of buffer fluids 1031, 1041, the high-density beads (generally without attached antibodies with fluorophores attached thereto) enter and cluster toward the bottom of the testing area 1030 in the closed end of the detection chamber 1050. Owing to the density of the antibody combinations being less than the density of the buffer fluid 1031, the antibody combinations (and the respectively attached delta-9-THC (Δ9)) are not able to pass into the testing area 1030 in the closed end portion of the detection chamber 1050, and are, accordingly, constrained by the buffer fluid 1031 to remain in the buffer fluid 1045 of the input area 1040.

A light source 1021, which can be located outside of but proximate to the testing area 1030 emits light 1022 directed towards the high-density beads. In contrast to the fluorescence of the fluorophores attached to the high-density bead combinations of FIG. 8, when the arrangement presented in FIG. 10 is stimulated by the electromagnetic energy of the light 1022, no light is emitted as there are few or no fluorophores present in the buffer fluid 1031. Hence, there is no fluorescence effect being captured and/or detected by detector 1023, and accordingly signal 1070 indicates no fluorescence was detected (e.g., the signal 1070 can have a value of 0=no fluorescence detected).

A magnitude of the signal 1070 may be used to determine that none, or a very small number of (e.g., a threshold quantity, such as less than 10-20%), fluorophore-labelled antibodies originally in the third additive 1015 are now located in the testing area 1030, and accordingly, the fluorophore-labelled antibodies are attached to the delta-9-THC (Δ9) molecules from the fluid sample 1003. Hence, a conclusion may be drawn that the magnetically pre-separated detection sample 1003 (and original fluid sample 910 of FIG. 9) had delta-9-THC (Δ9) molecules present, or a level of delta-9-THC molecules present that is above a given legal limit.

With reference to FIG. 10 in comparison to FIG. 8, a fluorophore-labelled antibody will preferentially bind with a ‘free’ Δ9 molecule from the magnetically pre-separated detection sample 1003, then it will with a delta-9-THC simulant molecule attached to the high-density beads. Hence, the preferential binding can be utilized to control where the fluorophore-labelled antibody will be within the detection chamber 1050 after application of the centrifugal force. For a magnetically pre-separated detection sample 1003 containing no, or few, delta-9-THC (Δ9) molecules present, after application of the centrifugal force, the fluorophore-labelled antibodies are located in the buffer fluid 1031 (e.g., attached to the high-density beads). For a magnetically pre-separated detection sample 1003 containing a high volume of Δ9 molecules, after application of the centrifugal force, the fluorophore-labelled antibodies are located in the buffer fluid 1041 (e.g., attached to the delta-9-THC (Δ9) molecules).

In various implementations, the method steps illustrated in FIGS. 7-10 and described in detail above may be modified to optimize the process and achieve specific targets.

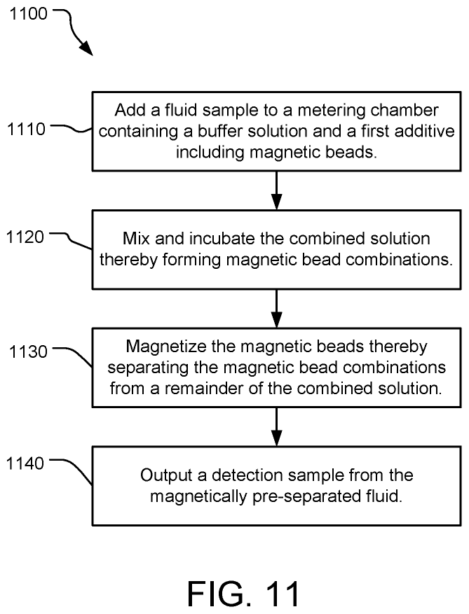

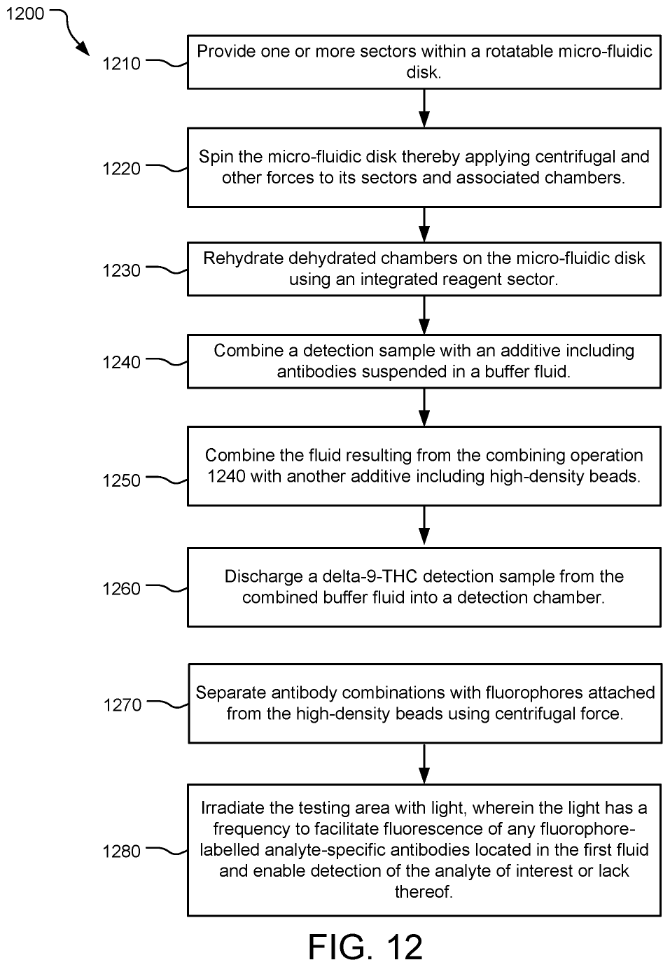

FIGS. 11-12 illustrate example methodologies relating to utilizing centrifugal force separation to detect presence of an analyte of interest in a fluid. In various implementations, the analyte is a delta-9-THC (Δ9) molecule(s), and the fluid is a saliva sample. While the methodologies are shown and described as being a series of acts that are performed in a sequence, it is to be understood and appreciated that the methodologies are not limited by the order of the sequence. For example, some acts can occur in a different order than what is described herein. In addition, an act can occur concurrently with another act. Further, in some instances, not all acts may be required to implement the methodologies described herein.

FIG. 11 is a flowchart of example operations 1100 for executing a magnetic pre-separation sequence on a fluid sample. An adding operation 1110 adds a fluid sample (e.g., a saliva sample) that may or may not contain delta-9-THC (Δ9) molecule(s) to a metering chamber. The fluid sample may also include various metabolites, including but not limited to delta-9-THC (Δ9) metabolites. The metering chamber includes an additive including magnetic beads, each with an antibody attached thereto, wherein the antibodies are specific to (e.g., binds to) the metabolites (e.g., delta-9-THC metabolites and other metabolites).

A mixing and incubation operation 1120 is performed on the combined solution that allows the magnetic beads with antibodies attached to attract the metabolites (e.g., carboxy (COOH), hydroxy (OH)). Delta-9-THC, if it were present, would remain unattached to the magnetic beads. Sufficient magnetic beads are included in the metering chamber to bond with substantially all the metabolites (M) from the fluid sample, thereby forming magnetic bead combinations. The mixed and incubated combined solution is output from the metering chamber into a magnetic separation chamber.

A magnetizing operation 1130 magnetizes the magnetic beads thereby separating the magnetic bead combinations from a remainder of the combined solution. A magnet is placed adjacent to a magnetic separation chamber that attracts the magnetic beads with the metabolites from the fluid sample now attached (magnetic bead combinations). As a result, the magnetic beads and attached metabolites, if present, are drawn to and cluster around the magnet, while the remaining components of the fluid (including Delta-9-THC, if present), remains substantially evenly suspended within the combined solution. As the metabolites are unwanted components for a further delta-9-THC detection sequence (see e.g., operations 1200 of FIG. 12), the metabolites are substantially separated from the combined solution. Alternative or additional to the magnetizing operation 1130, a density pre-separation operation may operate using density rather than magnetization to impart a similar pre-separation of unwanted components.

An outputting operation 1140 outputs a detection sample from the magnetically pre-separated fluid. As the metabolites are bonded to the magnetic beads and magnetically separated from the remainder of the combined solution, the detection sample contains delta-9-THC, if present, but no or few metabolites (M) thereof.

FIG. 12 is a flowchart of example operations 1200 for executing a delta-9-THC detection sequence. A providing operation 1210 provides one or more sectors within a rotatable micro-fluidic disk. Each of the sectors is fed by a fluid input (e.g., test subject fluid, control fluid, or liquid reagent fluid) and includes a series of chambers to execute a series of operations on fluid as it flows through the chambers on the disk in a generally parallel manner with other similar sectors on the disk. A spinning operation 1220 spins the rotatable disk thereby applying centrifugal and other forces to its sectors and associated chambers.

In some implementations, some components within the disk chambers (e.g., magnetic beads within a first chamber, a second additive within a third chamber, and a third additive within a fourth chamber) are initially in a de-hydrated state to maximize shelf life for long-term storage. A rehydration operation 1230 rehydrates some of the de-hydrated chambers using an integrated reagent sector. An integrated liquid reagent pack is opened, which ultimately feeds a reagent fluid distribution ring that lies outside of the other disk chambers with liquid reagent. The liquid reagent is then drawn radially inward into the other disk chambers to rehydrate empty or de-hydrated chambers therein, specifically detection chambers. Other empty or de-hydrated chambers may be filled using the liquid detection sample as it moves generally radially outward through those chambers.

A first combining operation 1240 combines the magnetically pre-separated sample obtained from outputting operation 1140 of FIG. 11 with an additive including antibodies suspended in a buffer fluid in anti-body mixing chamber(s). The antibodies each have regions specific to a delta-9-THC molecule (see also third additive 615 of FIG. 6) and a fluorophore attached thereto. The detection sample and the additive are mixed and allowed to incubate within the anti-body mixing chamber(s) for a period of time. During the incubation period, the antibodies within the additive bond with any delta-9-THC, if present, within the detection sample.

A second combining operation 1250 combines the fluid resulting from the first combining operation 1240 with another additive including high-density beads each with a first protein that is delta-9-THC specific and with a delta-9-THC simulant molecule attached thereto (see also first additive 605 of FIG. 6) in a high-density bead chamber(s). The additive is mixed with the other fluids and allowed to incubate for another period of time. During the incubation period, in the absence of actual delta-9-THC from the detection sample, the antibodies from the additive bond with the high-density beads with the delta-9-THC simulant from the additive forming high-density bead combinations. Alternatively, owing to delta-9-THC (Δ9) molecules being already attached to the antibodies within the additive, there are no (or a minimal number of) delta-9-THC specific antibodies remaining to attach to the high-density beads. Hence, there are few or none of the antibody fluorophores attached to the high-density beads and the high-density beads generally remain unattached to other molecules within the buffer fluid.

A discharging operation 1260 draws the fluid resulting from the second combining operation 1250 (a delta-9-THC detection sample) from the combined buffer fluid and deposits the delta-9-THC detection sample into a detection chamber. A separating operation 1270 separates the antibody combinations with fluorophores attached from the high-density beads using centrifugal force. Centrifugal forces generated by the spinning disk cause the high-density beads with the attached analyte molecules and any respective fluorophore-labelled analyte-specific antibodies attached thereto to pass through buffer fluid such that the first beads are displaced to an outer region of the detection chamber that serves as a testing area.

The centrifugal force also causes any fluorophore-labelled analyte-specific antibodies with attached analyte molecules to be displaced towards the first (higher-density) buffer fluid, however, owing to the density of the fluorophore-labelled analyte-specific antibodies with attached analyte molecules being less than the density of the first fluid, the fluorophore-labelled analyte-specific antibodies with attached analyte molecules are unable to pass through to the first fluid and the fluorophore-labelled analyte-specific antibodies with attached analyte molecules are constrained in the second (lower-density) buffer fluid.

An irradiation operation 1280 irradiates the testing area with light, wherein the light has a frequency to facilitate fluorescence of any fluorophore-labelled analyte-specific antibodies located in the testing area, wherein any fluorophore-labelled analyte-specific antibodies located in the testing area are bound to the analytes attached to the first beads. An absence of fluorescence from the testing area indicates a strong presence of delta-9-THC within the magnetically pre-separated sample. An increasing magnitude of fluorescence of the testing area defines an increasingly less prevalent delta-9-THC. The magnitude of fluorescence is based upon the amount of fluorophore-labelled analyte-specific antibodies located in the first fluid. Based upon the measured magnitude of fluorescence, a volume of analyte, delta-9-THC, present in the first fluid is determined, and further based thereon, a relative quantity of delta-9-THC molecules in the detection sample can be determined.

If a degree of fluorescence measured at the first fluid is below a threshold value, a determination can be made that the test fluid contains an amount of analyte that is above a defined limit (e.g., a legal limit). Accordingly, based thereon, a determination can be made that the test fluid was taken from an individual who was in a current state of legally defined intoxication and further determination of the degree of intoxication of the individual should be performed (e.g., back at a police station). In contrast, if a degree of fluorescence measured at the first fluid is above a threshold value, a determination can be made that the test fluid does not contain an amount of analyte that is above a defined limit (e.g., a legal limit). Accordingly, based thereon, a determination can be made that the test fluid was taken from an individual who was not in a current state of legally defined intoxication and the individual can be released from further testing.

The operations 1200 of FIG. 12 are discussed above with specific regard to delta-9-THC, though another analyte of interest may be substituted for the delta-9-THC with similar effects. Similarly, while use of a centrifugal micro-fluidic disk is discussed above to execute the operations of FIG. 12, a different mechanism, such as a laboratory, may be used to achieve similar effects.

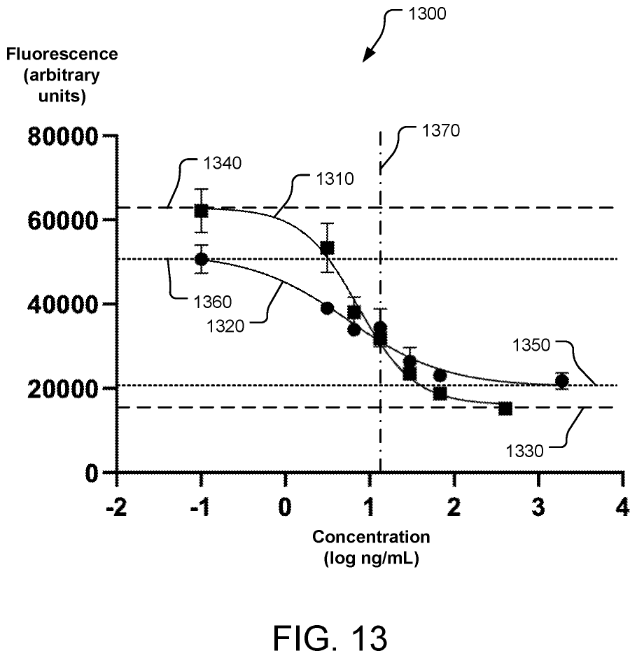

FIG. 13 presents an example graph 1300 of fluorescence (illustrated with arbitrary units) plotted against concentration (ng/mL) for two example analytes of interest (i.e., delta-9-THC and cocaine) obtained as a result of the detection sequences described herein. Plot 1310 is a curve fit to five known data points represented by squares using delta-9-THC as the analyte of interest. Plot 1320 is a curve fit to five known data points represented by circles using cocaine as the analyte of interest. Other analytes of interest, including but not limited to illicit drugs, may yield similar curves, and are contemplated herein.

Control sectors (e.g., control sectors 217, 219 of FIG. 2) within a micro-fluidic disk (e.g., micro-fluidic disk 200 of FIG. 2) are generally used to perform analyses on a control sample released by an integrated control fluid pack (e.g., integrated control fluid pack 204 of FIG. 2 for comparison against similar analyses performed on a test subject fluid sample by drug detection sectors (e.g., drug detection sectors 203, 205, 207, 209, 211, 213, 215 of FIG. 2) within the micro-fluidic disk. The control sectors 217, 219 are generally used to provide high and low points of reference, respectively, for determining relative quantities of any detected drugs or other chemicals that may be present in the test subject's fluid sample. While many variations of a curve fit may be applied to known data points to mathematically approximate the plots 1310, 1320, the following is an example method using known high and low measurements provided by the high and low control sectors of the micro-fluidic disk.

The plot 1310 is defined by a high concentration (or positive control) parameter (fTOP) at approximately 15,000 fluorescence units at greater than 2.7 log ng/ml of delta-9-THC and a low concentration (or negative control) parameter (fBOTTOM) at approximately 63,000 fluorescence units at approximately −1 log ng/ml of delta-9-THC, illustrated by dashed lines 1330, 1340, respectively. The plot 1320 is defined by a high concentration parameter at approximately 20,000 fluorescence units at greater than 3.2 log ng/ml of cocaine and a low concentration parameter at approximately 50,000 fluorescence units at approximately −1 log ng/ml of cocaine, illustrated by dotted lines 1350, 1360, respectively. In various implementations, the high and low concentration parameters are provided by the control sectors and a positive control and a negative control, respectively.

The plots 1310, 1320 may further be characterized by a midpoint parameter (fMID) illustrated by dot-dash line 1370 at approximately 1.1 log ng/ml of delta-9-THC or cocaine. While a common midpoint parameter is illustrated in FIG. 13 for both delta-9-THC and cocaine, in other implementations, the plots 1310, 1320 may have distinctly different midpoint parameters. While not explicitly illustrated, the plots 1310, 1320 may also each have a slope parameter (fSLOPE) that approximates the slope of the plots 1310, 1320 that exists between the high and low concentration parameters and that passes through the midpoint parameters.

In an example implementation, for a given set of reagents (e.g., high-density beads, fluorescent-bound antibodies, metabolite depletion, etc.) there exists a measurable response depending on the concentration of the analyte of the interest found in the assay. This response, the dosed response curve (illustrated by plots 1310, 1320), can be described by the aforementioned 4 separate numerical parameters: fTOP, fBOTTOM, fMID, fSLOPE. These 4 parameters can be uniquely found by at least 4 separate data points measured at differing compound concentrations. Once known, the 4 parameters can be used on an unknown sample to numerically calculate the compound concentration based on the system response.

Of the 4 parameters, the value of two may be highly dependent on a specific system sensitivity (PMT sensitivity, well targeting, optical aberrations and scattering, etc.). These two values are fTOP and fBOTTOM. The other two parameters, fMID and fSLOPE, may be assumed independent of system sensitivity. Thus, fMID and fSLOPE can be established at a factory for a given set of reagents, and then communicated to the system (e.g., via QR code, manual input, database lookup, etc.). The values of fTOP and fBOTTOM for an analyte of the interest can then be computed by checking the response for an undetectable concentration and a saturation concentration for the assay referred to herein as a negative control and a positive control, respectively.

Alternatively, if a system's specific sensitivity is established and controlled, fTOP and fBOTTOM can be determined based on previously determined parameters on a separate system and the relative sensitivity between the two systems. All such relevant parameters can then be communicated to the measuring system. Such a setup would not require the negative control and the positive control to establish fTOP and BOTTOM.

The logical operations of the present invention may be implemented (1) as a sequence of processor-implemented steps executed in one or more computer systems and (2) as interconnected machine or circuit modules within one or more computer systems. The implementation is a matter of choice, dependent on the performance requirements of the computer system implementing the invention. Accordingly, the logical operations making up the embodiments of the invention described herein are referred to variously as operations, steps, objects, or modules. Furthermore, the logical operations may be performed in any order, adding and omitting as desired, unless explicitly claimed otherwise or a specific order is inherently necessitated by the claim language.

The above specification, examples, and data provide a complete description of the structure and use of example implementations of the invention. Since many implementations of the invention can be made without departing from the spirit and scope of the invention, the invention resides in the claims hereinafter appended. Furthermore, structural features of the different implementations may be combined in yet another implementation without departing from the recited claims. The implementations described above, and other implementations are within the scope of the following claims.

What is claimed is:

1. A centrifugal micro-fluidic disk comprising:
an array of chemical detection sectors, each extending radially outward from a sample input port, each of the chemical detection sectors including:
a high-density bead chamber containing high-density beads each with a first protein that is specific to an analyte of interest, the high-density beads further with an analyte of interest simulant molecule attached thereto, the high-density bead chamber to mix a test subject sample received from the sample input port and antibodies having regions specific to an analyte of interest and a fluorophore attached thereto with the high-density beads; and
a detection chamber to receive the mixed test subject sample, antibodies, and high-density beads, wherein antibody combinations with fluorophores attached are separated from the high-density beads using a centrifugal force, and wherein an absence of fluorescence from the high-density beads indicates a presence of the analyte of interest within the test subject sample.

2. The centrifugal micro-fluidic disk of claim 1, wherein each of the chemical detection sectors further includes:
a metering chamber to receive the test subject sample from the sample input port and meter a quantity of the test subject sample into the high-density bead chamber.

3. The centrifugal micro-fluidic disk of claim 1, wherein each of the chemical detection sectors further includes:
an antibody mixing chamber to mix the test subject sample with the antibodies prior to entry into the high-density bead chamber.

4. The centrifugal micro-fluidic disk of claim 1, further comprising:
a first control sector extending outward from a control input port, the first control sector including:
a metering chamber to receive a control sample from the control input port, mix an analyte of interest as a positive control, and meter a quantity of the mixed positive control sample out of the metering chamber;
an antibody mixing chamber to mix the metered and mixed positive control sample with antibodies having regions specific to an analyte of interest and a fluorophore attached thereto;
a high-density bead chamber containing high-density beads each with a first protein that is specific to the analyte of interest, the high-density beads further with an analyte of interest simulant molecule attached thereto, the high-density bead chamber to mix the metered and mixed positive control sample and antibodies with the high-density beads; and
a detection chamber to receive the metered and mixed positive control sample, antibodies, and high-density beads, wherein antibody combinations with fluorophores attached are separated from the high-density beads using a centrifugal force, and wherein an absence of fluorescence from the high-density beads indicates a presence of the analyte of interest within the positive control sample.

5. The centrifugal micro-fluidic disk of claim 4, further comprising:
a second control sector extending outward from the control input port, the second control sector including:
a metering chamber to receive a negative control sample from the control input port and meter a quantity of the negative control sample out of the metering chamber;
an antibody mixing chamber to mix the metered negative control sample with antibodies having regions specific to the analyte of interest and a fluorophore attached thereto;
a high-density bead chamber containing high-density beads each with a first protein that is specific to the analyte of interest, the high-density beads further with an analyte of interest simulant molecule attached thereto, the high-density bead chamber to mix the metered negative control sample and antibodies with the high-density beads; and
a detection chamber to receive the metered negative control sample, antibodies, and high-density beads, wherein antibody combinations with fluorophores attached are separated from the high-density beads using a centrifugal force, and wherein fluorescence from the high-density beads indicates an absence of the analyte of interest within the negative control sample.

6. The centrifugal micro-fluidic disk of claim 5, wherein an intensity of fluorescence between a positive control parameter and a negative control parameter defines concentration of the analyte of interest within the test subject sample.

7. The centrifugal micro-fluidic disk of claim 6, wherein the first control sector establishes the positive control parameter and the second control sector establishes the negative control parameter.

8. The centrifugal micro-fluidic disk of claim 4, further comprising:
a single-use control fluid pack attached to the disk at the control input port, the single-use fluid pack to be opened by a user prior to using the disk to assay a test subject sample.

9. The centrifugal micro-fluidic disk of claim 1, wherein one of the array of chemical detection sectors is a delta-9-THC detection sector.

10. The centrifugal micro-fluidic disk of claim 1, wherein the array of chemical detection sectors includes one or more of cocaine, methamphetamine, and fentanyl detection sectors.

11. The centrifugal micro-fluidic disk of claim 1, wherein the array of chemical detection sectors includes one or more of oxandrolone, stanozolol, and erythropoietin detection sectors.

12. The centrifugal micro-fluidic disk of claim 1, wherein the array of chemical detection sectors includes one or more of E. coli O157:H7, Listeria spp., and Salmonella spp. detection sectors.

13. The centrifugal micro-fluidic disk of claim 1, wherein the array of chemical detection sectors includes one or more of Streptococcus pneumoniae, Respiratory Syncytial Virus, and COVID-19 detection sectors.

14. The centrifugal micro-fluidic disk of claim 1, wherein the array of chemical detection sectors includes human or veterinary health diagnostic detection sectors.

15. The centrifugal micro-fluidic disk of claim 9, wherein the metering chamber for the delta-9-THC detection sector includes magnetic beads, each of the magnetic beads including an antibody attached thereto, wherein the antibody is specific to metabolites of delta-9-THC, wherein the magnetic beads and metabolites of delta-9-THC form magnetic bead combinations, further comprising:
a magnetic separation chamber to receive the magnetic beads, delta-9-THC, and magnetic bead combinations from the metering chamber; and
a magnet to separate the magnetic beads and magnetic bead combinations from the delta-9-THC within the magnetic separation chamber, wherein an output of the magnetic separation chamber to the antibody mixing chamber excludes the magnetic beads and magnetic bead combinations.

16. The centrifugal micro-fluidic disk of claim 1, wherein the metering chambers for one or more of the detection sectors include pre-separation beads, each of the pre-separation beads including an antibody attached thereto, wherein the antibody is specific to metabolites of the analyte of interest, wherein the pre-separation beads and metabolites of the analyte of interest form pre-separation bead combinations, such ones of the detection sectors further comprising:
a separation chamber to receive the pre-separation beads, delta-9-THC, and magnetic bead combinations from the metering chamber and separate the pre-separation bead combinations from the analyte of interest within the separation chamber, wherein an output of the separation chamber to the antibody mixing chamber excludes the pre-separation beads and pre-separation bead combinations.

17. The centrifugal micro-fluidic disk of claim 1, wherein the array of chemical detection sectors are each to assay the test subject sample for a different chemical compound.

18. A centrifugal micro-fluidic disk comprising:
an integrated liquid reagent sector including:
a single-use reagent fluid pack attached to the disk at a liquid reagent input port;
a reagent fluidic circuit extending radially outward from the liquid reagent input port; and
an reagent distribution ring positioned radially outward from the reagent fluidic circuit; and
an array of sample detection networks, each extending radially outward from a sample input port, wherein:
in response to an opening of the single-use reagent fluid pack and rotation of the disk, the reagent distribution ring receives reagent moving radially outward from the reagent fluidic circuit and feeds the reagent radially inward into the sample detection networks to fill the detection chambers until the reagent within the sample detection networks achieves a pressure equilibrium with the reagent within the reagent fluidic circuit.

19. The centrifugal micro-fluidic disk of claim 18, wherein the reagent further rehydrates one or more dehydrated components within the chambers of the sample detection networks.

20. The centrifugal micro-fluidic disk of claim 18, wherein the reagent fluidic circuit includes a reagent mixing chamber that includes dehydrated reagent, and wherein the single-use reagent fluid pack contains a liquid to be mixed with the dehydrated reagent in the reagent mixing chamber.

21. The centrifugal micro-fluidic disk of claim 18, wherein the reagent contained within the single-use reagent fluid pack is Percoll.

22. A method of detecting an analyte of interest within a fluid sample comprising:
combining the fluid sample including the analyte of interest and one or more metabolites of the analyte of interest with a first additive including magnetic beads within a first chamber, each of the magnetic beads including an antibody attached thereto, wherein the antibody is specific to the metabolites;
forming magnetic bead combinations of the magnetic beads and the metabolites;
outputting the combined fluid sample and first additive from the first chamber to a second chamber;
attracting the magnetic beads and the magnetic bead combinations to a magnet applied to an exterior of a second chamber; and
outputting a magnetically pre-separated sample of the fluid sample and the first additive from the second chamber.

23. The method of claim 22, wherein the magnetically pre-separated sample includes the analyte of interest but less than a threshold quantity of the metabolites found in the fluid sample.

24. The method of claim 22, further comprising:
combining the magnetically pre-separated sample with a second additive including second antibodies within a third chamber, each of the second antibodies having regions specific to the analyte of interest and a fluorophore attached thereto;
forming antibody combinations of the second antibodies and molecules of the analyte of interest within the third chamber;
discharging the antibody combinations of the second antibodies and molecules of the analyte of interest from the third chamber to a fourth chamber;
combining the antibody combinations of the second antibodies and molecules of the analyte of interest with a third additive containing high-density beads each with a first protein that is analyte of interest specific and with an analyte of interest simulant molecule attached thereto within the fourth chamber;
discharging the antibody combinations and the third additive from the fourth chamber to a fifth chamber;
separating the antibody combinations with fluorophores attached from the high-density beads using a centrifugal force applied to the fifth chamber; and
detecting an absence of fluorescence from the high-density beads indicating presence of the analyte of interest within the magnetically pre-separated sample.

25. The method of claim 22, wherein the magnetically pre-separated sample includes neither the analyte of interest nor the metabolites found in the fluid sample.

26. The method of claim 22, further comprising:
combining the magnetically pre-separated sample with a second additive including second antibodies within a third chamber, each of the second antibodies having regions specific to the analyte of interest and a fluorophore attached;|

discharging the magnetically pre-separated sample and the second additive from the third chamber to a fourth chamber;
combining magnetically pre-separated sample and the second additive with a third additive containing high-density beads each with a first protein that is analyte of interest specific and with an analyte of interest simulant molecule attached thereto within the fourth chamber;
forming high-density bead combinations of the second additive and the third additive within the fourth chamber;
discharging the magnetically pre-separated sample, the second additive, and the third additive, including the high-density bead combinations, from the fourth chamber to a fifth chamber;
separating the high-density bead combinations with fluorophores attached using a centrifugal force applied to the fifth chamber;
detecting fluorescence from the high-density beads indicating an absence of the analyte of interest within the magnetically pre-separated sample.

27. The method of claim 22, wherein the analyte of interest is delta-9-THC and the metabolites are that of delta-9-THC.

28. The method of claim 22, performed on a centrifugal micro-fluidic disk.

29. The method of claim 24, wherein one or more of the magnetic beads within the first chamber, the second additive within the third chamber, and the third additive within the fourth chamber are in a dehydrated state, further comprising:
rehydrating one or more of the magnetic beads within the first chamber, the second additive within the third chamber, and the third additive within the fourth chamber by flowing the fluid sample through one or more of the first, third, and fourth chambers.
